(19) United States
(12) Patent Application Publication
(10) Pub. No.: US 2013/0310269 A1
(43) Pub. Date: Nov. 21, 2013

(54) HOT-START DIGITAL PCR

(71) Applicant: Austin So, Pleasanton, CA (US)

(72) Inventor: Austin So, Pleasanton, CA (US)

(21) Appl. No.: 13/886,708

(22) Filed: May 3, 2013

Related U.S. Application Data
(60) Provisional application No. 61/643,120, filed on May 4, 2012.

(51) Int. Cl. C12Q 1/68

(52) U.S. Cl. CPC C12Q 1/6853; C12Q 1/6858
USPC 506/9; 435/91.2; 435/6.12; 536/24.33

(57) ABSTRACT

The present disclosure provides methods and compositions for performing nucleic acid reactions, such as hot-start digital polymerase chain reaction (dPCR).

### CROSS-REFERENCE

This application claims the benefit of U.S. Patent Application No. 61/643,120, filed May 4, 2012, which application is herein incorporated by reference in its entirety.

### BACKGROUND

Detection and quantification of specific nucleic acid sequences using PCR can be useful in a large and diverse range of research and clinical applications. The first generation of PCR users performed end-point analysis by gel electrophoresis to obtain qualitative results. The advent of real-time PCR spawned a second generation that enabled quantification by monitoring the progression of amplification after each cycle using fluorescence probes. In real-time PCR, quantitative information can be obtained from the cycle threshold (C_{T}), a point on the analogue fluorescence curve where the signal increases above background. External calibrators or normalization to endogenous controls can be required to estimate the concentration of an unknown. Imperfect amplification efficiencies affect C_{T} values which in-turn can limit the accuracy of this technique for absolute quantification.

Digital PCR can combine limiting dilution, end-point PCR, and Poisson statistics to yield an absolute measure of nucleic acid concentration. In digital PCR, target DNA molecules can be distributed across multiple replicate reactions at a level where some reactions have no template and others have one or more template copies. After amplification to the terminal plateau phase of PCR, reactions containing one or more templates can yield positive end-points, whereas those without template can remain negative.

Currently, three approaches can be used by commercially available digital PCR systems. A first approach uses microwells or microfluidic chambers to split the sample into hundreds of nanoliter partitions. A second approach, called BEAMing, can be based on emulsion PCR, where templates are clonally amplified in the presence of beads. Post-PCR, the emulsion is broken to recover the beads, which can be subsequently labeled with a fluorescent hybridization probe and read by conventional flow-cytometry. A third approach uses water-in-oil droplets as the enabling technology to realize high-throughput digital PCR in a low-cost and practical format. This approach can take advantage of simple microfluidic circuits and surfactant chemistries to divide a small volume (e.g., 20 μL) mixture of sample and reagents into a large number (e.g., ˜20,000) monodisperse droplets (i.e., partitions). An automated droplet flow-cytometer can read each set of droplets after PCR.

PCR has been used to detect target polynucleotide sequences of interest in test samples. One example is the measurement of genetic variations of single nucleotide polymorphisms (SNPs) between members of a species. SNPs are one of the most common types of genetic variation. A SNP is a single base pair mutation at a specific locus, usually consisting of two alleles (where the rare allele frequency is >1%). SNPs are found to be involved in the etiology of many human diseases and are becoming of particular interest in pharmacogenetics. Because SNPs are conserved during evolution, they have been proposed as markers for use in quantitative trait loci (QTL) analysis and in association studies in place of microsatellites. SNPs can also provide a genetic fingerprint for use in identity testing.

### SUMMARY

Provided herein are methods and compositions for hot-start PCR that can be carried out, e.g., in droplets, e.g., as digital droplet PCR.

In one aspect, a method for amplifying a target polynucleotide is provided, comprising: (a) contacting a target polynucleotide with a reaction mixture comprising: (i) a first forward primer that is complementary to a first sequence of said target polynucleotide and comprises: a first modified nucleoside residue at a position corresponding to a target locus residing within said first sequence, a first fluorophore that is attached to said first forward primer and is upstream of said target locus, and a first quencher that is attached to said first forward primer and is downstream of said target locus; (ii) a first reverse primer; and (iii) a digestive enzyme that is capable of cleaving said first forward primer when said modified nucleoside residue matches a first allele at said target locus, and is not capable of cleaving said first forward primer when said modified nucleoside residue does not match said first allele at said target locus; and (b) amplifying said target nucleotide in said reaction mixture with a DNA polymerase, thereby obtaining a plurality of amplicons. In some cases, the first modified nucleoside comprises a ribonucleoside. In some cases, the first modified nucleoside comprises a 2′-fluoro-modified RNA nucleoside. In some cases, the first modified nucleoside comprises a 2′-fluoro-modified DNA nucleoside.

In some cases, the first forward primer further comprises a blocking residue located downstream of said target locus. In some cases, the blocking residue comprises an inverted base. In some cases, the blocking residue locates about 1 to 10 bases from said target locus. In some cases, the blocking residue locates between said target locus and the residue to which said quencher is attached. In some cases, the blocking residue locates about 1 to 10 bases from said quencher.

In some cases, the digestive enzyme comprises RNase H2. In some cases, the digestive enzyme is thermostable. In some cases, the DNA polymerase substantially lacks 5′-nuclease activity.

In some cases, the method further comprises detecting said amplicons. In some cases, the detecting is carried out in real-time. In some cases, the detecting comprises a melting curve analysis.

In some cases, the amplifying step is carried out by performing digital PCR. In some cases, the digital PCR is microfluidic-based digital PCR. In some cases, the digital PCR is droplet digital PCR. In some cases, the digital PCR is performed in droplets having a volume that is between about 1 pL and about 100 nL.

In some cases, the first modified residue matches said first allele. In some cases, the first modified residue does not match said first allele. In some cases, the first reverse primmer comprises (i) a modified residue, and (ii) a blocking group. In some cases, the reaction mixture comprises a second forward primer comprising: a second modified nucleoside residue at a position corresponding to said target locus, a second fluorophore that is attached to said second forward primer and is upstream of said target locus, and a second quencher that is attached to said second forward primer and is downstream of said target locus, wherein said second modified nucleoside residue matches a second allele of said target locus. In some cases, the reaction mixture comprises two to four forward primers, each comprising: a modified nucleoside residue at a position corresponding to said target locus, a fluorophore that is attached to each of said forward primers and is upstream of said target locus, and a quencher that is attached to each of said forward primers and is downstream of said target locus, wherein said modified nucleoside residue of each of said forward primers matches a different allele of said target locus. In some cases, the detecting has a sensitivity of 1/100 to 1/1,000, as defined by mutant/(mutant+wild-type).

In another aspect, a primer is provided that is complementary to a first sequence of a target polynucleotide, comprising: (a) a first modified nucleoside residue at a position corresponding to a target locus residing within said first sequence; (b) a fluorophore attached to said primer and is upstream of said target locus; and (c) a quencher attached to said first primer and is downstream of said target locus.

In some cases, the first modified nucleoside comprises a ribonucleoside. In some cases, the first modified nucleoside comprises a 2′-fluoro-modified RNA nucleoside. In some cases, the first modified nucleoside comprises a 2′-fluoro-modified DNA nucleoside. In some cases, the first forward primer further comprises a blocking residue located downstream of said target locus. In some cases, the blocking residue comprises an inverted base. In some cases, the blocking residue locates about 1 to 10 bases from said target locus. In some cases, the blocking residue locates between said target locus and the residue to which said quencher is attached. In some cases, the blocking residue locates about 1 to 10 bases from said quencher.

In another aspect, a kit is provided comprising a primer described herein.

In another aspect, a method for amplifying a target polynucleotide is provided, comprising: (a) contacting a target polynucleotide with a reaction mixture comprising: (i) a first forward primer that is complementary to a first sequence of the target polynucleotide and comprises: a first modified nucleoside residue at a position corresponding to a target locus residing within the first sequence, a first fluorophore that is coupled to the first forward primer and 5′ of the position corresponding to the target locus, and a first quencher that is coupled to the first forward primer and is 3′ of the position corresponding to the target locus; (ii) a first reverse primer; and (iii) a digestive enzyme that is capable of cleaving the first forward primer when the modified nucleoside residue matches a first allele at the target locus, and is not capable of cleaving the first forward primer when the modified nucleoside residue does not match the first allele at the target locus; and (b) amplifying the target nucleotide in the reaction mixture with a DNA polymerase, thereby obtaining a plurality of amplicons.

In some cases, the first modified nucleoside comprises a ribonucleoside. In some cases, the first modified nucleoside comprises a 2′-fluoro-modified RNA nucleoside. In some cases, the first modified nucleoside comprises a 2′-fluoro-modified DNA nucleoside.

In some cases, the first forward primer further comprises a blocking residue located 3′ of the position corresponding to the target locus. In some cases, the blocking residue comprises an inverted base. In some cases, the blocking residue locates about 1 to about 10 bases from the position corresponding to the target locus. In some cases, the blocking residue locates between the position corresponding to the target locus and the residue to which the first quencher is coupled. In some cases, the blocking residue locates about 1 to about 10 bases from the first quencher.

In some cases, the digestive enzyme comprises RNase H2. In some cases, the digestive enzyme is thermostable. In some cases, the DNA polymerase substantially lacks 5′-nuclease activity.

In some cases, the method further comprises detecting the amplicons. In some cases, the detecting is carried out in real-time. In some cases, the detecting comprises a melting curve analysis. In some cases, the amplifying comprises performing digital PCR. In some cases, the digital PCR is microfluidic-based digital PCR. In some cases, the digital PCR is droplet digital PCR. In some cases, the digital PCR is performed in droplets each having a volume that is between about 1 pL and about 100 nL.

In some cases, the first modified nucleoside residue matches the first allele. In some cases, the first modified nucleoside residue does not match the first allele. In some cases, the first reverse primer comprises (i) a modified residue, and (ii) a blocking group. In some cases, the first fluorophore is directly attached to the first forward primer. In some cases, the first quencher is directly attached to the first forward primer.

In some cases, the reaction mixture further comprises a second forward primer comprising: a second modified nucleoside residue at a position corresponding to the target locus, a second fluorophore that is coupled to the second forward primer and is 5′ of the position corresponding to the target locus, and a second quencher that is coupled to the second forward primer and is 3′ of the position corresponding to the target locus, wherein the second modified nucleoside residue matches a second allele of the target locus. In some cases, the second fluorophore is directly attached to the second forward primer, and the second quencher is directly attached to the second forward primer. In some cases, the second allele is different from the first allele.

In some cases, the reaction mixture comprises two to four forward primers, each comprising: a modified nucleoside residue at a position corresponding to the target locus, a fluorophore that is coupled to each of the forward primers and is 5′ of the position corresponding to the target locus, and a quencher that is coupled to each of the forward primers and is 3′ of the position corresponding to the target locus, wherein the modified nucleoside residue of each of the forward primers matches a different allele of the target locus. In some cases, the detecting has a sensitivity of 1/100 to 1/1,000, as defined by mutant/(mutant+wild-type).

In another aspect, a nucleic acid molecule for detecting a sequence of a target polynucleotide is provided, comprising: (a) a primer that is complementary to said sequence, wherein said primer comprises a first modified nucleoside residue at a position corresponding to a target locus residing within the sequence; (b) a fluorophore coupled to the primer, wherein said fluorophore is 5′ of the position corresponding to the target locus; and (c) a quencher coupled to the first primer, wherein said quencher is 3′ of the position corresponding to the target locus.

In some cases, the first modified nucleoside comprises a ribonucleoside. In some cases, the first modified nucleoside comprises a 2′-fluoro-modified RNA nucleoside. In some cases, the first modified nucleoside comprises a 2′-fluoro-modified DNA nucleoside. In some cases, the primer further comprises a blocking residue located 3′ of the position corresponding to the target locus. In some cases, the blocking residue comprises an inverted base. In some cases, the blocking residue locates about 1 to about 10 bases from the position corresponding to the target locus. In some cases, the blocking residue locates between the position corresponding to the target locus and the residue to which the quencher is coupled. In some cases, the blocking residue locates about 1 to about 10 bases from the quencher.

In another aspect, a kit is provided comprising a nucleic acid molecule described herein.

In another aspect, a method is provided comprising: (a) contacting a target polynucleotide with a reaction mixture comprising: (i) a first forward primer that is complementary to a first sequence of the target polynucleotide and comprises: a first modified nucleotide residue at a position corresponding to a target locus residing within the first sequence, and a blocking group; (ii) a reverse primer; and (iii) a digestive enzyme that is capable of cleaving the first forward primer when the modified nucleoside residue matches a first allele at the target locus, and is not capable of cleaving the first forward primer when the modified nucleoside residue does not match the first allele at the target locus; and (b) amplifying the target nucleotide in the reaction mixture with a DNA polymerase if the modified nucleoside residue of the first forward primer matches the first allele at the target locus, thereby obtaining a plurality of amplicons.

In some cases, the first modified nucleoside comprises a ribonucleoside. In some cases, the first modified nucleoside comprises a 2′-fluoro-modified RNA nucleoside. In some cases, the first modified nucleoside comprises a 2′-fluoro-modified DNA nucleoside.

In some cases, the blocking group is located 3′ of the position corresponding to the target locus. In some cases, the blocking group comprises an inverted base. In some cases, the blocking group locates about 1 to about 10 bases from the position corresponding to the target locus.

In some cases, the digestive enzyme comprises RNase H2. In some cases, the digestive enzyme is thermostable.

In some cases, the method further comprises detecting the amplicons. In some cases, the detecting is carried out in real-time. In some cases, the detecting comprises a melting curve analysis. In some cases, the detecting comprises use of a probe. In some cases, the probe anneals to an amplicon 3′ of the target locus. In some cases, the probe comprises a fluorophore/quencher pair. In some cases, the probe is modified at its 5′ end such that cannot be incorporated into an amplicon. In some cases, the detecting comprises use of a dye.

In some cases, the amplifying comprises performing digital PCR. In some cases, the digital PCR is microfluidic-based digital PCR. In some cases, the digital PCR is droplet digital PCR. In some cases, the digital PCR is performed in droplets each having a volume that is between about 1 pL and about 100 nL.

In some cases, the first modified nucleoside residue matches the first allele. In some cases, the first modified nucleoside residue does not match the first allele. In some cases, the first reverse primer comprises (i) a modified residue, and (ii) a blocking group.

In some cases, the method further comprises (a) contacting a target polynucleotide comprising the target locus with a second reaction mixture comprising (i) a second forward primer that differs from the first forward primer in that it comprises a sequence that differs from that of the first forward primer at the position corresponding to the target locus, (ii) the reverse primer; and (iii) a digestive enzyme that is capable of cleaving the second forward primer when the modified nucleoside residue matches a second allele at the target locus, and is not capable of cleaving the second forward primer when the modified nucleoside residue does not match the second allele at the target locus; and (b) amplifying the target nucleotide in the reaction mixture with a DNA polymerase if the modified nucleoside residue of the second forward primer matches the second allele at the target locus, thereby obtaining a plurality of amplicons.

### INCORPORATION BY REFERENCE

All publications, patents, and patent applications mentioned in this specification are herein incorporated by reference in their entirety for all purposes, to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages will be obtained by reference to the following detailed description that sets forth illustrative cases, in which the principles are utilized, and the accompanying drawings of which:

FIG. 1 depicts hot-start PCR primers recognizing mutant or wild-type residues in a template nucleic acid.

FIG. 2 depicts a method of identifying a mutant or wild-type residue in a template nucleic acid.

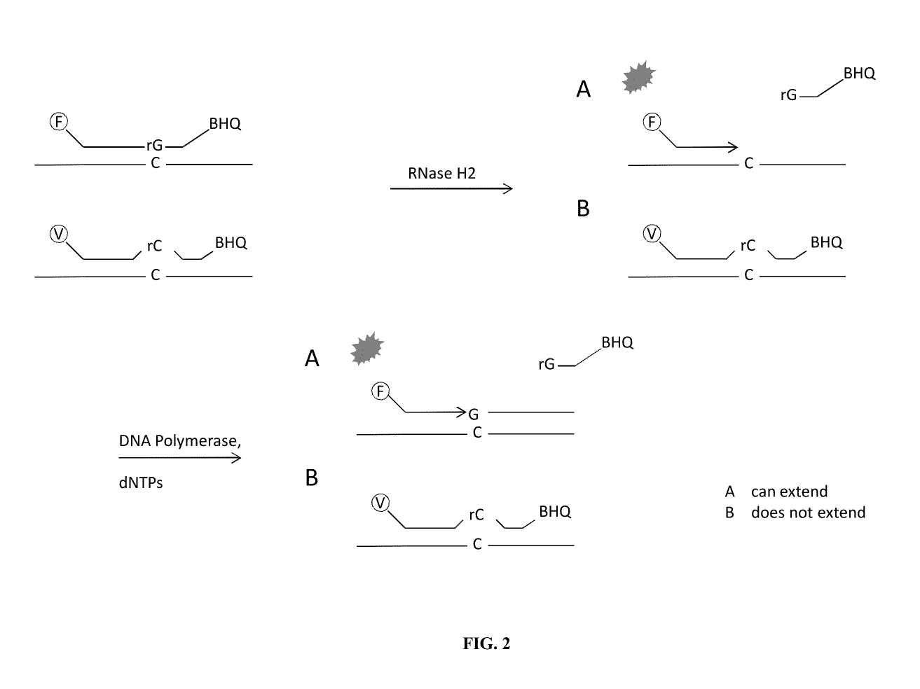

FIG. 3 depicts an exemplary work-flow of the PCR using hot-start primers to detect SNP in a target polynucleotide.

FIG. 4 illustrates another embodiment for identifying an allele in a template nucleic acid.

FIG. 5 illustrates an embodiment of a method provided herein for multiplexing.

### DETAILED DESCRIPTION

Several aspects are described below with reference to example applications for illustration. It should be understood that numerous specific details, relationships, and methods are set forth to provide a full understanding of the methods and compositions described herein. One having ordinary skill in the relevant art, however, will readily recognize that the methods described herein can be practiced without one or more of the specific details or with other methods. The methods described herein are not limited by the illustrated ordering of acts or events, as some acts can occur in different orders and/or concurrently with other acts or events. Furthermore, not all illustrated acts or events are required to implement a methodology in accordance with the methods described herein.

The terminology used herein can be for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which can depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, or within 2-fold, of a value. Where particular values are described in the application and claims, unless otherwise stated the term “about” meaning within an acceptable error range for the particular value should be assumed.

### I. Methods for Hot-Start PCR

### A. Overview

Methods and compositions are provided herein for carrying out reactions to amplify a target polynucleotide comprising a target locus with a pair of primers, one of which can be a “hot-start” primer. A hot-start primer can comprise a modified residue or moiety that can enable the detection of an allele at the target locus.

In general, a hot-start primer can comprise a modified residue (e.g., a ribonucleoside), a blocking group, and a pair of fluorophore/quencher flanking the modified residue. The fluorophore can be located at the 5′ side of the modified residue, and the blocking group and the quencher can be located at the 3′ side of the modified residue. The blocking group can prevent the extension of the primer by a polymerase. The quencher can also function as a blocking group, and in such case there can be no additional blocking group. Each of the two primers can have a sequence that is complementary to the target polynucleotide. A digestive enzyme can recognize and cleave the primer if the modified residue matches (hybridizes to) the sequence of the target polynucleotide. The cleavage can release the quencher (and an additional blocking group, if present, from the hot-start primer). Removal of the blocking group can enable primer extension by polymerase, and the release of the quencher can lead to emission of fluorescence, which can indicate the occurrence of target polynucleotide amplification. In some cases, if the modified residue does not match the allele at the target locus, the primer can not be cleaved by the enzyme, or has a reduced ability to be cleaved by the enzyme. The presence of a blocking group (or quencher if the quencher also functions as a blocking group) can prevent primer extension and the fluorophore can remain quenched, which can indicate no target polynucleotide amplification has occurred. Therefore, the presence or absence of detectable signal from the fluorophore can correlate with the occurrence of amplification as well as the allele at the target locus. In some cases, an increase in a detectable signal of a fluorophore can correlate with the occurrence of amplification as well as the allele at the target locus.

In one aspect, the present disclosure provides a method for amplifying a target polynucleotide, comprising: (a) contacting a target polynucleotide with a reaction mixture comprising: (i) a first forward primer that is complementary to a first sequence of the target polynucleotide and comprises: a first modified nucleoside residue at a position corresponding to a target locus residing within the first sequence, a first fluorophore coupled to the first forward primer and 5′ of said target locus, and a first quencher coupled to the first forward primer and 3′ of the target locus; (ii) a first reverse primer; and (iii) a digestive enzyme that is capable of cleaving the first forward primer when the modified nucleoside residue matches a first allele at the target locus, and is not capable of (or has a reduced capacity of) cleaving the first forward primer when the modified nucleoside residue does not match the first allele at the target locus; and (b) amplifying the target nucleotide in the reaction mixture with a DNA polymerase, thereby obtaining a plurality of amplicons. In some cases, the method further comprises contacting the target polynucleotide with a second, third, or fourth forward primer, each of which differs from the first forward primer in that it (i) comprises a sequence that differs from that of the first forward primer at the target locus and (ii) comprises a fluorophore that differs from the fluorophore coupled to the first forward primer. Optionally, the method further comprises detecting at least one fluorophore. The method can also comprise identifying genetic variations (e.g., SNPs) based on the detecting of the at least one fluorophore. In some cases, a first, second, third, and/or fourth primer are in the same reaction mixture.

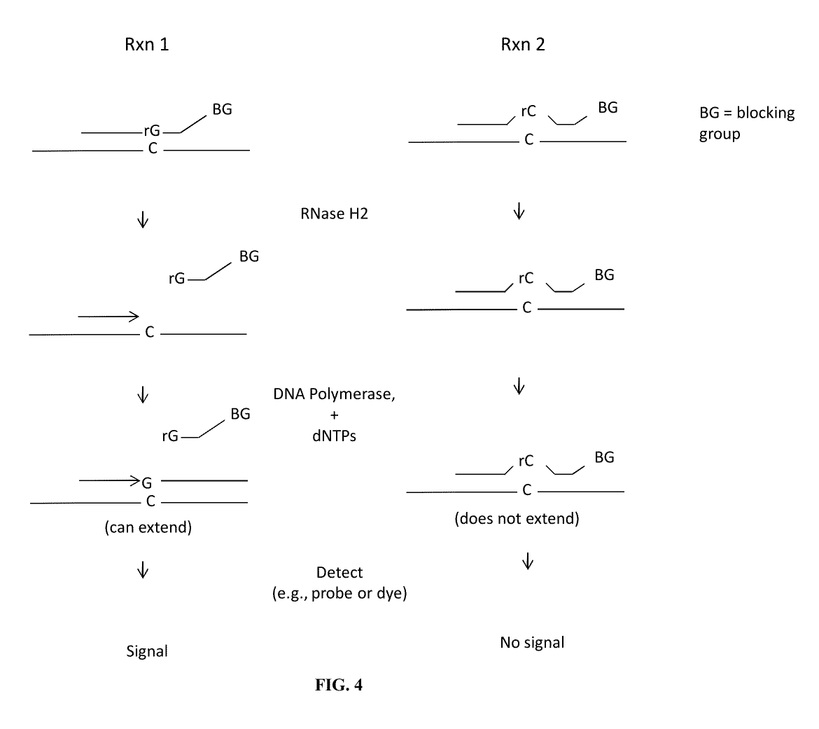

In some cases, one or more forward primers comprising a fluorophore/quencher pair are not used in the methods described herein. For example, in some cases, a first forward primer comprises a modified nucleotide at a residue corresponding to a target locus, a blocking group, and the forward primer does not comprise a fluorophore/quencher pair. A second, third, and/or fourth primer pair can be used that differ from the first forward primer and from each other by the identity of the modified nucleotide at the residue corresponding to a target locus. The first, second, third, and/or fourth forward primers can be used in different reaction mixtures to generate amplicons, and the amplicons can be detected using, e.g., a common probe or a dye. The identity of the target locus can be determined by knowing the identity of the forward primer in each reaction mixture. (See e.g., FIG. 4).

In some cases, a method is provided comprising: (a) contacting a target polynucleotide with a reaction mixture comprising: (i) a first forward primer that is complementary to a first sequence of the target polynucleotide and comprises: a first modified nucleotide residue at a position corresponding to a target locus residing within the first sequence, and a blocking group; (ii) a reverse primer; and (iii) a digestive enzyme that is capable of cleaving the first forward primer when the modified nucleoside residue matches a first allele at the target locus, and is not capable of (or has a reduced capacity of) cleaving the first forward primer when the modified nucleoside residue does not match the first allele at the target locus; and (b) amplifying the target nucleotide in the reaction mixture with a DNA polymerase if the modified nucleoside residue matches the first allele at the target locus, thereby obtaining a plurality of amplicons.

In some cases, the method further comprises contacting a target polynucleotide comprising the target locus with a second reaction mixture comprising (i) a second forward primer that differs from the first forward primer in that it comprises a sequence that differs from that of the first forward primer at the position corresponding to the target locus, (ii) the reverse primer; and (iii) a digestive enzyme that is capable of cleaving the second forward primer when the modified nucleoside residue matches a second allele at the target locus, and is not capable of (or has a reduced capacity of) cleaving the second forward primer when the modified nucleoside residue does not match the second allele at the target locus; and (b) amplifying the target nucleotide in the reaction mixture with a DNA polymerase if the modified nucleoside residue of the second forward primer matches the second allele at the target locus, thereby obtaining a plurality of amplicons.

In some cases, the method further comprises contacting a target polynucleotide comprising the target locus with a third reaction mixture comprising (i) a third forward primer that differs from the first and second forward primer in that it comprises a sequence that differs from that of the first and second forward primer at the position corresponding to the target locus, and (ii) the reverse primer; and (iii) a digestive enzyme that is capable of cleaving the third forward primer when the modified nucleoside residue matches a third allele at the target locus, and is not capable of (or has a reduced capacity of) cleaving the third forward primer when the modified nucleoside residue does not match the third allele at the target locus; and (b) amplifying the target nucleotide in the reaction mixture with a DNA polymerase if the modified nucleoside residue of the third forward primer matches the third allele at the target locus, thereby obtaining a plurality of amplicons.

In some cases, the method further comprises contacting a target polynucleotide comprising the target locus with a fourth reaction mixture comprising (i) a fourth forward primer that differs from the first, second, and third forward primer in that it comprises a sequence that differs from that of the first, second, and third forward primer at the position corresponding to the target locus, and (ii) the reverse primer and (iii) a digestive enzyme that is capable of cleaving the fourth forward primer when the modified nucleoside residue matches a fourth allele at the target locus, and is not capable of (or has a reduced capacity of) cleaving the fourth forward primer when the modified nucleoside residue does not match the fourth allele at the target locus; and (b) amplifying the target nucleotide in the reaction mixture with a DNA polymerase if the modified nucleoside residue of the fourth forward primer matches the fourth allele at the target locus, thereby obtaining a plurality of amplicons.

In some cases, e.g., where a first, second, third, and/or fourth forward primer are provided in separate reactions mixtures, the plurality of amplicons from the separate reaction mixtures can be detected with a probe that anneals to the amplified target polynucleotide. In some cases, the probe anneals 3′ of the target locus in the amplicon. In some cases, the probe comprises a fluorophore and a quencher. The probe can be nuclease sensitive. In some cases, the probe can form a hairpin when not annealed to the target polynucleotide. The probe can be blocked at the 5′ end of the probe to prevent incorporation of the probe into an amplicon; such a probe can be used for melting curve analysis.

In some cases, e.g., where a first, second, third, and/or fourth primer are provided in separate reaction mixtures, the plurality of amplicons can be detected with a dye or stain, e.g., an intercalating dye. Examples of intercalating dyes include ethidium bromide, propidium iodide, acridine orange, or 9-amino-6-chloro-2-methoxyacridine (ACMA), In some cases, the dye is a cyanine dye, e.g., SYBR Green, SYBR green II, SYBR Gold, YO (Oxazole Yellow), TO (Thiazole Orange), or PG (PicoGreen).

The term “coupled to,” as used herein, can generally mean “attached to” either directly or through one or more intermediaries. For example, a first species (e.g., atom or molecule) coupled to a second species can be directly attached to the second species. As another example, the first species can be coupled to the second species through at least a third species.

The method provided herein can provide benefits beyond simple SNP detection. Because the primer and probe are combined, the potential footprint of the assay can be reduced, e.g., to about 40 bp. This property can maximize the signal from degraded samples (FFPEs) or plasma/urine samples.

In some cases, the methods and compositions provided herein can result in an increase in specificity as compared to methods and compositions comprising Taqman probes. The improved specificity can be due, in part, to competitive hybridization between a perfectly matched primer and a mismatched primer, as well as to the specificity of an enzyme (e.g., RNase H2) for the perfectly matched primer, resulting in extension of only the perfectly matched primer. Specificity can be determined by two factors: the relative difference in binding energy between the perfect and mismatch probes with the target; and, the recognition of the perfect match primer by the digesting enzyme, which can recognize an RNA base, a methylated base, a hairpin, etc.

### B. Target Polynucleotide

Methods provided here can be directed to the amplification and detection of target nucleic acid molecule.

A “target nucleic acid molecule,” “target molecule,” “target polynucleotide,” “target polynucleotide molecule” or grammatically equivalents thereof can be any nucleic acid of interest. In one aspect, target nucleic acids are genomic nucleic acids. DNA derived from the genetic material in the chromosomes of a particular organism can be genomic DNA. Target nucleic acids can include naturally occurring or genetically altered or synthetically prepared nucleic acids (such as genomic DNA from a mammalian disease model). Target nucleic acids can be obtained from virtually any source and can be prepared using methods known in the art. For example, target nucleic acids can be directly isolated without amplification using methods known in the art, including without limitation, extracting a fragment of genomic DNA from an organism (e.g., a cell or bacteria) to obtain target nucleic acids. A target polynucleotide can also encompass cDNA generated from RNA (such as mRNA, non-coding RNA, microRNA, siRNA, ribosomal RNA, tRNA, catalytic RNA, viral RNA) through RT-PCR.

An “oligonucleotide” or “polynucleotide” or grammatical equivalents can refer to at least two nucleotides covalently linked together. “Nucleic acid” or grammatical equivalents can refer to either a single nucleotide or at least two nucleotides covalently linked together. A nucleic acid can contain phosphodiester bonds, although in some cases, as outlined below (for example in the construction of primers and probes such as label probes), nucleic acid analogs can have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid (also referred to herein as “PNA”) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (also referred to herein as “LNA”), (Koshkin et al., J. Am. Chem. Soc. 120.13252 3 (1998)); positive backbones (Denpcy et al., Proc. Natl. Acad. Sci. USA 92:6097 (1995)); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside &amp; Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35. “Locked nucleic acids” are also included within the definition of nucleic acid analogs. LNAs are a class of nucleic acid analogues in which the ribose ring is “locked” by a methylene bridge connecting the 2′-O atom with the 4′-C atom. All of these references are hereby expressly incorporated by reference. These modifications of the ribose-phosphate backbone can be done to increase the stability and half-life of such molecules in physiological environments. For example, PNA:DNA and LNA-DNA hybrids can exhibit higher stability and thus can be used in some cases. The target nucleic acids can be single stranded or double stranded, as specified, or contain portions of both double stranded or single stranded sequence. Depending on the application, the nucleic acids can be DNA (including genomic and cDNA), RNA (including mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 2,6-diaminopurine, thiouracil etc.

In general, the position of the nucleoside residue being modified can correspond to a target locus of the target polynucleotide. A “target locus” or “locus of interest” can be one or more positions of a sequence of a target polynucleotide to be detected and/or analyzed. In some cases, a target polynucleotide comprises about, more than, less than, or at least 1, 2, 3, 4, 5, 6, 7, 8, 9, 10, 11, 12, 13, 14, 15, 16, 17, 18, 19, or 20 target loci. In some cases, a target locus comprises about, more than, less than, or at least 1, 2, 3, 4, 5, 6, 7, 8, 9, 10, 11, 12, 13, 14, 15, 16, 17, 18, 19, or 20 nucleotides. In some cases, a target locus is in a coding region of a target polynucleotide; in some cases, a target locus is not in a coding region of a target polynucleotide.

In some cases, a target locus to be analyzed is a genetic marker. A “genetic marker” can be a gene or DNA sequence with a known location on a chromosome that can be used to identify cells, individuals, or species. A genetic marker can be a variation (which can arise due to mutation or alteration in the genomic loci) that can be observed. A genetic marker can be a short DNA sequence, such as a sequence surrounding a single base-pair change (single nucleotide polymorphism, SNP), or a long one, like minisatellites. In some cases, a target polynucleotide comprises about, more than, less than, or at least 1, 2, 3, 4, 5, 6, 7, 8, 9, 10, 11, 12, 13, 14, 15, 16, 17, 18, 19, or 20 genetic markers.

SNPs can be markers for a number of phenotypic traits, non-limiting examples of which include disease propensity and severity, wellness propensity, drug responsiveness, and susceptibility to adverse drug reactions. In some cases of the present disclosure, the identification of a specific SNP in an individual—coupled with knowledge of the association of the SNP with a phenotypic trait—enables the design of diagnostic, preventative, prognostic, and/or therapeutic applications that improve disease management and/or enhance understanding of disease states. In some cases, such applications facilitate the discovery of more effective treatments, such as personalized treatment regimens. In some cases, a target polynucleotide comprises about, more than, less than, or at least 1, 2, 3, 4, 5, 6, 7, 8, 9, 10, 11, 12, 13, 14, 15, 16, 17, 18, 19, or 20 SNPs. In some cases, a SNP is biallelic. In some cases, a SNP is triallelic.

A number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental methods), protocols, and conditions used to assay the variation, and can include information associating a SNP with a particular phenotypic trait.

### C. Primers

A target polynucleotide can be amplified with a pair of primers.

A “primer” can be an oligonucleotide or nucleic acid capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature. Primer extension can also be carried out in the absence of one or more of the nucleotide triphosphates in which case an extension product of limited length can be produced. In some cases, the polynucleotide template is a DNA molecule. In some cases, the polynucleotide template is an RNA molecule. In some cases, primers described herein are detectably labeled. In some cases, primers are not detectably labeled. Primers can be hot-start or regular primers. Hot-start primers (or primers with modified residue(s)) are described herein. The terms “regular primer” and “non-hot-start primer” can refer to primers that are not hot-start primers as defined herein. Primers described as “for,” “directed to,” or “capable of amplifying” a particular target sequence can be complementary to the ends of the target sequence, with the 3′ ends facing inward, such that the target sequence can be amplified in a PCR reaction.

Generally, a pair of primers can be used in the amplification reaction; one can be a forward primer and one can be a reverse primer. The forward primer can be a hot-start primer as provided herein that comprises a modified residue that can be recognized by a cleavage enzyme (or digestive enzyme), and the modified residue can be flanked by a fluorophore/quencher pair. A reverse primer can be a regular primer, i.e., not modified and not comprising a fluorophore/quencher or a blocking group.

In some cases, the reverse primer is also a hot-start primer, and comprises a modified residue that matches the corresponding position at the target polynucleotide, and a blocking group. The reverse primer can be cleaved only at an elevated temperature to release the blocking group, thus enabling primer extension and amplification. The use of a pair of hot-start primers can enable specific amplification. In some cases, a reverse primer does not comprise a fluorophore/quencher pair.

The sequence of the length of the reverse primer can depend on the sequence of a target polynucleotide and a target locus. The reverse primer can be designed such that it is suitable for amplification in conjunction with the cleaved forward primer. For example, the length and/or Tm of the reverse primer can be optimized. In some cases, if the reverse primer is a regular primer, it is shorter than the full length forward primer. If the reverse is also a hot-start primer, then the primers can be designed so that the cleaved forward primer and cleaved forward primer are suitable for amplification reactions.

A “nucleotide,” “nucleoside,” “nucleotide residue,” and “nucleoside residue” can be a deoxyribonucleotide or ribonucleotide residue, or other similar nucleoside analogues capable of serving as components of primers suitable for use in an amplification reaction (e.g., PCR reaction). Such nucleoside and derivatives thereof can be used as the building blocks of the primers described herein, except where indicated otherwise. In some cases, nucleoside derivatives or bases that have been chemical modified to enhance their stability or usefulness are used in an amplification reaction. In some cases, the chemical modification does not interfere with their recognition by DNA polymerase as deoxyguanine, deoxycytosine, deoxythymidine, or deoxyadenine, as appropriate.

A primer can be a single-stranded DNA prior to binding a template polynucleotide. In some cases, a primer is initially double-stranded. The appropriate length of a primer depends on the intended use of the primer but can range from about 6 to about 50 nucleotides, or about 15 to about 35 nucleotides. In some cases, a primer is about, more than, less than, or at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases. A short primer molecule can generally use cooler temperatures to form sufficiently stable hybrid complexes with a template. A primer need not reflect the exact sequence of a template nucleic acid, but can be sufficiently complementary to hybridize with the template. The design of suitable primers for the amplification of a given target sequence is well known in the art and described in the literature cited herein.

Primers can incorporate additional features which can allow for the detection or immobilization of the primer but do not alter the basic property of the primer, that of acting as a point of initiation of DNA synthesis. For example, primers can contain an additional nucleic acid sequence at the 5′ end which does not hybridize to the target nucleic acid, but which facilitates cloning or detection of the amplified product. The region of the primer which is sufficiently complementary to the template to hybridize can be referred to herein as the hybridizing region. In some cases, a 5′ region of a primer that does not anneal to a target polynucleotide is about, more than, less than, or at least 1, 2, 3, 4, 5, 6, 7, 8, 9, 10, 11, 12, 13, 14, 15, 16, 17, 18, 19, or 20 bases.

In some cases, nucleotide analogues that can be used in methods and compositions described herein include derivatives wherein the sugar is modified, as in 2′-O-methyl, 2′-deoxy-2′-fluoro, and 2′,3′-dideoxynucleoside derivatives, nucleic acid analogs based on other sugar backbones, such as threose, locked nucleic acid derivatives, bicyclo sugars, or hexose, glycerol and glycol sugars, nucleic acid analogs based on non-ionic backbones, such as “peptide nucleic acids,” these nucleic acids and their analogs in non-linear topologies, such as dendrimers, comb-structures, and nanostructures, and these nucleic acids and their analogs carrying tags (e.g., fluorescent, functionalized, or binding) bound to their ends, sugars, or nucleobases.

One non-limiting example of non-classical nucleotide analogues suitable for use in methods and compositions described herein are locked nucleic acid (LNA) nucleotide analogues. Some embodiments of LNA nucleotide analogues are bicyclic nucleic acid analogs that contain one or more 2′-O, 4′-C methylene linkages, which effectively lock the furanose ring in a C3′-endo conformation. This methylene linkage “bridge” restricts the flexibility of the ribofuranose ring and locks the structure into a rigid bicyclic formation. Oligonucleotides comprising LNA nucleotide analogues can demonstrate a much greater affinity and specificity to their complementary nucleic acids than do natural DNA counterparts. LNAs can hybridize to complementary nucleic acids even under adverse conditions, such as under low salt concentrations. LNA nucleotide analogues are commercially available, and are described, inter alia, in U.S. Pat. Nos. 6,130,038, 6,268,490, and 6,670,461.

Another non-limiting example of non-classical nucleotide analogues suitable for use in methods and compositions described herein are peptide nucleic acid (PNA) nucleotide analogues. In some embodiments of PNA nucleotide analogues, the negatively charged sugar-phosphate backbone of DNA can be replaced by a neutral polyamide backbone composed of N-(2-aminoethyl) glycine units (see illustration below, wherein B represents a nucleoside base). The chemical configuration of PNA typically enables the nucleotide bases to be positioned in approximately the same place as in natural DNA, allowing PNA to hybridize with complementary DNA or RNA sequence. PNA nucleotide analogues are commercially available, and are described in WO 92/20702, WO 92/20703 and WO 93/12129.

Another non-limiting example of non-classical nucleotide analogues suitable for use in methods and compositions described herein are glycol nucleic acid (GNA) nucleotide analogues (Zhang, L et al (2005) A simple glycol nucleic acid. J. Am, Chem. Soc. 127:4174-4175).

Another non-limiting example of non-classical nucleotide analogues suitable for use in methods and compositions described herein are threose nucleic acid (TNA) nucleotide analogues (Wu et al, Organic Letters, 2002, 4(8):1279-1282).

Another non-limiting example of non-classical nucleotide analogues suitable for use in methods and compositions described herein are bicyclic and tricyclic nucleoside analogs (Steffens et al, Helv Chim Acta (1997) 80:2426-2439; Steffens et al, J Am Chem Soc (1999) 121: 3249-3255; Renneberg et al, J Am Chem Soc (2002) 124: 5993-6002; and Renneberg et al, Nucl Acids Res (2002) 30: 2751-2757).

Another non-limiting example of non-classical nucleotide analogues suitable for use in methods and compositions described herein are phosphonomonoester nucleic acids which incorporate a phosphorus group in the backbone, for example analogues with phosphonoacetate and thiophosphonoacetate internucleoside linkages (US Pat. App. No. 2005/0106598; Sheehan et al, Nucleic Acids Res (2003); 31(14):4109-18). In other cases, a cyclobutyl ring replaces the naturally occurring furanosyl ring.

In other cases of non-classical nucleotide analogues suitable for use in methods and compositions described herein, the base is modified. A representative, non-limiting list of modified nucleobases includes 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), and pyridoindole cytidine (H-pyrido(3′,′: 4,5)pyrrolo[2,3-d]pyrimidin-2-one). Modified nucleobases can also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in the art as suitable for increasing the binding affinity of the compounds described herein. These include 5-substituted pyrimidines, 6-azapyrimidines and N-2, N-6 and O-6 substituted purines, including 2-aminopropyladenine, 5-propynyluracil and 5-propynylcytosine. Modified nucleobases and their use are described, in U.S. Pat. No. 3,687,808, U.S. Pat. No. 4,845,205; U.S. Pat. No. 5,130,302; U.S. Pat. No. 5,134,066; U.S. Pat. No. 5,175,273; U.S. Pat. No. 5,367,066; U.S. Pat. No. 5,432,272; U.S. Pat. No. 5,457,187; U.S. Pat. No. 5,459,255; U.S. Pat. No. 5,484,908; U.S. Pat. No. 5,502,177; U.S. Pat. No. 5,525,711; U.S. Pat. No. 5,552,540; U.S. Pat. No. 5,587,469; U.S. Pat. No. 5,594,121, U.S. Pat. No. 5,596,091; U.S. Pat. No. 5,614,617; U.S. Pat. No. 5,645,985; U.S. Pat. No. 5,830,653; U.S. Pat. No. 5,763,588; U.S. Pat. No. 6,005,096; U.S. Pat. No. 5,681,941; and U.S. Pat. No. 5,750,692.

Another non-limiting example of non-classical nucleotide analogues suitable for use in methods and compositions described herein are polycyclic heterocyclic compounds in place of one or more of the naturally-occurring heterocyclic base moieties. A number of tricyclic heterocyclic compounds have been previously reported. These compounds can be used in antisense applications to increase the binding properties of the modified strand to a target strand. Modifications can be targeted to guanosines; hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications can hybridize with complementary guanine and the latter can hybridize with adenine and enhance helical thermal stability by extended stacking interactions (also see U.S. Publications 2003/0207804 and 2003/0175906).

In another embodiment, a primer utilized in methods and compositions described herein can comprise one or more universal nucleosides. Non-limiting examples of universal nucleosides are 5-nitroindole and inosine, as described in U.S. Patent Application Pub. Nos. 2009/0325169 and 2010/0167353.

Primers can be designed according to known parameters for avoiding secondary structures and self-hybridization. Different primer pairs can anneal and melt at about the same temperatures, for example, within about 1, 2, 3, 4, 5, 6, 7, 8, 9 or 10° C. of another primer pair. In some cases, at least, greater than, less than, or about 1, 2, 3, 4, 5, 6, 7, 8, 9, 10, 15, 20, 25, 30, 35, 40, 45, 50, 100, 200, 500, 1000, 5000, 10,000, 20,000, 30,000, 40,000, 50,000, 60,000, 70,000, 80,000, 90,000 or 100,000 primers or primer pairs are initially used. Such primers can hybridize to the genetic targets described herein.

Primers can be prepared by a variety of methods including but not limited to cloning of appropriate sequences and direct chemical synthesis using methods well known in the art (Narang et al., Methods Enzymol. 68:90 (1979); Brown et al., Methods Enzymol. 68:109 (1979)). Primers can also be obtained from commercial sources. The primers can have an identical melting temperature. The lengths of the primers can be extended or shortened at the 5′ end or the 3′ end to produce primers with desired melting temperatures. One of the primers of a primer pair can be longer than the other primer. The 3′ annealing lengths of the primers, within a primer pair, can differ. Also, the annealing position of each primer pair can be designed such that the sequence and length of the primer pairs yield the desired melting temperature. An equation for determining the melting temperature of primers smaller than 25 base pairs is the Wallace Rule (Td=2(A+T)+4(G+C)). Computer programs can also be used to design primers. The Tm (melting or annealing temperature) of each primer can be calculated using software programs. The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers can be incorporated into the products from each loci of interest; thus the Tm can be recalculated based on both the sequences of the 5′ half and the 3′ half of each primer.

C1. Hot-Start Primers with Modified Residues

In another aspect, described herein are primers with modified residue (modified primer) or Hot-Start Primers.

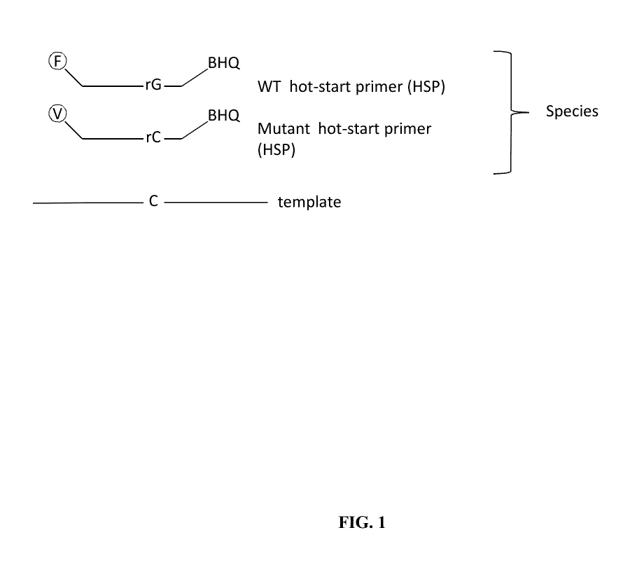

In general, a forward primer can be a hot-start primer that is substantially complementary to a first sequence of a target polynucleotide and can comprise a fluorophore/quencher pair. The forward primer can comprise a modified nucleoside residue at a position corresponding to a target locus residing within the first sequence of the target polynulcoetide. In some cases, the modified nucleoside residue can match (hybridize to) a given allele at the target locus. In some cases, the modified nucleoside residue does not match (hybridize to) a given allele at the target locus. When the modified nucleoside residue matches a first allele at the target locus, it can be recognized by a digestive enzyme, which can cleave the first primer. In some cases, when the modified nucleoside residue does not match a given allele at the target locus, it is not recognized by a digestive enzyme, which does not cleave the first primer. For example, assume the target locus is a SNP site that has an allele C. See FIG. 1. When the forward primer comprises a ribo-G which matches the C, it can be recognized and cleaved by a digestive enzyme (e.g., RNase H2). This process can lead to the removal of a blocking group on the first primer (in this case, the quencher) and the extension of the first primer by a polymerase, as well as a detectable signal emitted from the fluorophore due to removal of the quencher. When the forward primer comprises a ribo-C which does not match (hybridize to) the C, it is not recognized and cleaved by the digestive enzyme (e.g., RNase H2), and hence, there is no primer extension. In this way, the identity of the allele of the target locus can be determined by the signal generated by the de-quenched fluorophore. A plurality of forward primers labeled with different color of fluorophore can be used together in a multiplex reaction to determine all possible alleles (e.g., 1, 2, 3, or 4 for a SNP) at any given target loci. These forward primers can have one or more different modified residues (e.g., A, C, G, T) at the position corresponding to the target locus, and thus can match any of the four possible alleles at the target locus.

A “hot-start primer” or “modified primer” can be a primer that is inactive, or has reduced activity, until exposure to elevated temperatures. In general, hot-start primers can contain a modification so as to generate an extendible primer only when hybridized to complementary sequence while at elevated temperatures. One exemplary, non-limiting sub-class of this type of hot-start primers, referred to herein collectively as “ribo-primers,” are those that can be reversibly chemically modified such that they can be unable to serve as primers for DNA polymerase. Ribo-primers can also be substrates, when hybridized to a DNA target sequence, for cleavage by an endonuclease, for example an RNaseH2 endonuclease, which can result in removal of the chemical modification, as exemplified herein. In some cases, such modified primers can comprise an RNA moiety, which can serve as the RNase H cleavage site. Various embodiments of ribo-primers are further described herein.

In general, a hot-start primer can comprise DNA residues and one more modified residues. A hot-start primer can be any oligonucleotide that is extensible by a DNA polymerase (DNA, DNA+RNA, DNA+RNA+LNA, etc) and has a cleavage site that is specific for the perfect match species. A “modified residue” can be a residue that can match (hybridize) with one of the alleles (A, C, G, T) at the target locus of the target polynucleotide, and thus forms a matched base pair recognizable by a digestive enzyme which can lead to the cleavage of the primer by the digestive enzyme. In some cases, if the modified reside does not match (hybridize to) an allele of the target locus, it will not lead to the cleavage of the primer by the digestive enzyme. The modified residue can be a DNA nucleoside analogue, an RNA nucleoside, or a modified RNA nucleoside. In some cases, the modified residue comprises a RNA nucleoside or a modified RNA nucleoside. In some cases, the modified nucleoside of the hot-start primers comprises a ribonucleoside.

In some cases, a forward primer comprises DNA residues and one or more modified residues and a blocking group. The blocking group can be located 3′ to the cleavage site of an RNaseH2 endonuclease. In some cases, a forward primer does not comprise a fluorophore and/or quencher.

A blocking modification (alternatively referred to herein as an “inactivating chemical modification” or “blocking group”) of a ribo-primer can be located 3′ (downstream) to the cleavage site of an RNaseH2 endonuclease. The blocking modification can be located at the 3′-end of the ribo-primer. In some cases, the blocking modification can be positioned up to 1, 2, 3, 4, 5, 6, 7, 8, 9 or 10, 11, 12, 13, 14, 15, 16, 17, 18, 19, or 20 residues from the 3′ end of an oligonucleotide, followed by nucleotide sequence that is not either mostly non-complementary or entirely non-complementary to the target. In some cases, the blocking modification can be positioned up to 1, 2, 3, 4, 5, 6, 7, 8, 9, 10, 11, 12, 13, 14, 15, 16, 17, 18, 19, or 20 residues from the 3′ end of an oligonucleotide, followed by nucleotide sequence that is either mostly complementary or entirely complementary to the target. In any case, a “ribo-primer” as defined herein can contain a modification that can preclude its suitability as a primer for elongation and that can be reversed by the action of an endonuclease (also referred to herein as an “digestive enzyme” or “activating enzyme”). The reversal can involve physical removal of the modification.

In some cases, the term “ribo-primers” refers to variants of standard DNA oligonucleotide primers, containing the following modifications: At least one RNA residue is added at the 3′-end of a standard DNA oligonucleotide primer, followed by at least one, two, three, 4, 5, 6, 7, 8, 9, 10, 11, 12, 13, 14, 15, 16, 17, 18, 19, 20 or more additional DNA residues and a blocking group. The primer sequence formed by the RNA and DNA residues of the ribo-primers can exhibit partial or complete complementarity with the sequence of the target polynucleotide. A single RNA residue within a ribo-primer bound to a DNA target can be sufficient to render the heteroduplex a substrate for endonuclease cleavage by RNaseH2, and the cleavage can occur immediately 5′ to the RNA residue, removing the RNA residue itself and all residues and modifications 3′ to the RNA residue. In some cases, the presence within the ribo-primer of about, more than, less than, or at least one, 2, 3, 4, 5, 6, 7, 8, 9, 10 additional complementary RNA residues positioned 3′ to the first RNA residue will not affect the ability of the primer to serve as a substrate for endonuclease cleavage by RNaseH2.

In some cases, a ribo-primer used in a method or composition described herein contains a single 2′-fluoro-modified residue in an internal position thereof. Cleavage by RNase H2 typically occurs on the 5′ side of the 2′-fluoro-modified residue. See U.S. Pat. Appl. Pub. No. 20110086354, which is incorporated by reference for all purposes. In some cases, the ribo-primer comprises an RNase H2 cleavage domain comprising two adjacent 2′-fluoro-modified residues. In these cases, cleavage occurs primarily between the 2′-fluoro-modified residues. In some cases, the one or more 2′-fluoro-modified residues are DNA residues. In other cases, the one or more 2′-fluoro-modified residues are RNA residues. Each possibility can represent a separate embodiment.

In some cases, the modified nucleoside comprises a 2′-fluoro-modified RNA nucleoside. In some cases, the modified nucleoside comprises a 2′-fluoro-modified DNA nucleoside.

Other types of modified residues can also be present in a ribo-primer, including but not limited to 2′-O-alkyl-modified residues, e.g., 2′-O-methyl-modified residues, locked nucleic acids (LNA), 2′-ENA residues (ethylene nucleic acids), 2′-alkyl-modified residues, 2′-amino-modified residues, and 2′-thio-modified residues. In some cases, these modifications can be made to residues other than the RNA residue at the intended site of enzymatic cleavage, either immediately adjacent to the RNA residue or at positions further removed therefrom. In other cases, these modified residues can be introduced in place of the RNA residue at the intended site of enzymatic cleavage. In one embodiment, a 2′-fluoro-modified RNA residue is used in combination with a 2′ LNA-modified RNA residue.

In some cases, the 2′-hydroxy group of an RNA residue is replaced with one of the above-described alternative functional groups. In other cases, the phosphate group immediately 3′ to the RNA residue in the intended RNase H cleavage site is replaced with a nuclease-resistant linkage to prevent aberrant cleavage by an RNase H enzyme. In other cases, the oligonucleotide is modified with a nuclease-resistant linkage or residue further 3′ from the 3′-phosphate group of the RNA residue or on the 5′-side of the RNA residue. In some cases, the nuclease-resistant linkage that is utilized is selected from a phosphorothioate, phosphorodithioate, methylphosphonate, or boronate linkage. In another embodiment, an abasic residue such as a C3 spacer is inserted immediately 3′ to the RNA residue to prevent aberrant cleavage. Alternatively one or both of the hydrogen atoms on the 5′ carbon of the adjacent residue can be replaced with bulkier substituents such as methyl groups to inhibit background cleavage of a ribonucleotide residue. Combinations of these various modifications can also be employed. The above modifications are well-known in the art. Typically, such modifications are most useful for PCR reactions used for mismatch discrimination, such as allele-specific PCR (AS-PCR).

In some cases, alternative divalent cations such as Mn²⁺, Ni²⁺ or Co²⁺, with or without Mg²⁺, are incorporated into an assay buffer indicated for use with a ribo-primer. In certain cases, when such alternative divalent cations are present, enhanced cleavage by RNase H2 is achieved. In one more specific embodiment, Mn²⁺ in combination with Mg²⁺ is included in a buffer intended for use with a ribo-primer containing two adjacent 2′-fluoro-modified residues.

Various embodiments of ribo-primers, including the embodiments mentioned herein, are described in US App. Pub. Nos. 20110086354, 20090325169 and 20100167353, all incorporated herein by reference for all purposes.

The position of a ribonucleotide or other modified residue can affect the ability of a ribo-primer to serve as a substrate for RNase H2. In some cases, the ribonucleotide or other modified residue of a ribo-primer used in methods and compositions described herein is flanked on its 3′-side by a total of about 1 to about 10 DNA residues. In some cases, the ribonucleotide or other modified residue of a ribo-primer is flanked on its 3′-side by about, more than, at least, or less than 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 DNA residues. In other cases, a total of about 3 to about 6 DNA residues flank the ribonucleotide or other modified residue on its 3′-side. In some cases, a total of about 2 to about 5 DNA residues flank the ribonucleotide or other modified residue on its 3′-side. In some cases, a total of about 3 to about 5 DNA residues flank the ribonucleotide or other modified residue on its 3′-side. In some cases, a total of about 4 to about 5 DNA residues flank the ribonucleotide or other modified residue on its 3′-side. In some cases, about 4 to about 5 DNA residues that are complementary to the target sequence immediately flank the ribonucleotide or other modified residue on its 3′-side. Optionally, one or more additional DNA residues not complementary to the target sequence can be present 3′ to these about 4 to about 5 complementary DNA residues.

In some cases, about, less than, at least, or more than 8, 9, 10, 11, 12, 13, 14, 15, 16, 17, 18, 19, or deoxyribonucleotides that are complementary to the target sequence flank the ribonucleotide or other modified residue on its 5′-side. In some cases, about 8 to about 50, about 10 to about 50, about 12 to about 50, about 14 to about 50, about 16 to about 50, about 18 to about 50, about 20 to about 50, about 10 to about 20, about 10 to about 15, or about 15 to about 25 deoxyribonucleotide residues that are complementary to the target sequence flank the modified residue on its 5′-side. Optionally, one or more additional DNA residues not complementary to the target sequence can be present 5′ to these complementary DNA residues that are located 5′ to the modified residue.

In some cases, about, less than, at least, or more than 8, 9, 10, 11, 12, 13, 14, 15, 16, 17, 18, 19, or deoxyribonucleotides that are complementary to the target sequence flank the ribonucleotide or other modified residue on its 3′-side. In some cases, about 8 to about 50, about 10 to about 50, about 12 to about 50, about 14 to about 50, about 16 to about 50, about 18 to about 50, about 20 to about 50, about 10 to about 20, about 10 to about 15, or about 15 to about 25 DNA residues that are complementary to the target sequence flank the modified residue on its 3′-side. Optionally, one or more additional DNA residues not complementary to the target sequence can be present 3′ to these complementary DNA residues that are located 3′ to the modified residue.

In general, the length of the forward primer (hot-start primer) comprising the modified residue can be about 20 to about 50 nucleotides long, about 25 to about 40 nucleotides long, or about 25 to about 35 nucleotides long. In some cases, the length of the forward (hot-start primer) is about, more than, at least, or less than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. The Tm of the primer can be about 70° C. prior to cleavage and about 65° C. post-cleavage. The hot-start primer can have a sequence that is complementary to the sequence of the target polynucleotide, except the position of the modified residue, which in some cases matches the target polynucleotide, and in some cases does not match the target polynucleotide. The sequence and the length of the forward primer (hot-start primer) can depend on the sequence of the target polynucleotide and the target locus. In some cases, the forward primer (hot-start primer) is capable of hybridizing to the target polynucleotide at a suitable temperature to be recognized by the digestive enzyme prior to cleavage, and is still capable of hybridizing specifically to the target polynucleotide after cleavage, if cleavage occurs, to enable primer extension and amplification of the target polynucleotide.

In another apsect, the present disclosure provides methods to detect the presence of different alleles at the same target locus in a same reaction. In some cases, the reaction mixture of the amplification reaction comprises a second forward primer comprising: a second modified nucleoside residue at a position corresponding to the target locus residing within the first sequence, a second fluorophore coupled to the second forward primer and is 5′ of the position corresponding to the target locus, and a second quencher coupled to the second forward primer and is 3′ of the position corresponding to the target locus, wherein the second modified nucleoside residue matches a second allele of the target locus. In general, the second fluorophore can emit a detectable signal that is distinguishable from the signal emitted by the first fluorophore.

In some cases, the reaction mixture comprises one, two, three, or four forward primers, each comprising: a modified nucleoside residue at a position corresponding to a target locus residing within the first sequence, a fluorophore coupled to each of the forward primers and is 5′ of the position corresponding to the target locus, and a quencher coupled to each of the forward primers and is 3′ of the position corresponding to the target locus, wherein said modified nucleoside residue of each forward primer matches a different allele of said target locus. In general, each of the fluorophores emits a detectable signal that is distinguishable from the signal emitted by other fluorophores.

Thus, in these methods, two or more alleles at the same target locus can be detected simultaneously. If there is more than one target polynucloetide present in each reaction mixture (such as in a non-digital PCR reaction), more than one color of signal can be detected, indicating the presence of heterozygosity at the target locus. If there is only one target polynucleotide present in each reaction mixture (such as in a digital PCR reaction), then only one color of signal can be detected, indicating the presence of the allele corresponding to the hot-start primer directed to that allele. Thus, if the sample is homozygous, the same color signal is detected in all the reactions in digital PCR when a target polynucleotide is present. If the sample is heterozygous, then a different color signal is detected in different reactions in digital PCR when a target polynucleotide is present. By counting the number of different reactions detected of different color, the ratio of different alleles can be calculated.

In some cases, there is more than one target polynucleotide present in each partition of digital PCR. In these cases, the conditions for droplet generation can be controlled such that there is a very low chance that there is more than one target polynucleotide present.

In another aspect, the present disclosure provides multiplex methods to detect the presence of different alleles at different target loci in a same reaction, either as bulk PCR or digital PCR. Thus a plurality of pairs of forward primers and reverse primers can be used in each reaction mixture, each pair directed to different target loci and each of the forward primers can be labeled with different color. In some cases, the different forward primers target different alleles at the same locus and are labeled with a different color for different alleles.

In some cases, a plurality of forward primers are used in combination with a single reverse primer, each of the forward primer is directed to different target loci or different alleles of the same locus, and each of the forward primers are labeled with different colors.

In some cases, multiplexing of mutant versus wild-type species are performed so that all species can be quantified from a single sample.

In some cases, multiplex reactions are carried out with a plurality of pairs of forward primers and reverse primers, each directed to different target polynucleotides and each forward primer is labeled with different color of fluorophore.

In some cases, multiplexing of different target polynucleotides is performed so that all species can be quantified from a single sample.

In some cases, a first, second, third, and/or fourth forward primers are used in separate reaction mixtures with the same or different reverse primer to detect, e.g., one or more alleles at a target locus. For example, the first, second, third, and/or fourth primers can be complementary to a first sequence of a target polynucleotide, and each primer can comprise a modified nucleoside at a position corresponding to a target locus residing within the first sequence, and the primers can differ from each other by the identity of the modified nucleoside at the position corresponding to the target locus. The first, second, third, and/or fourth forward primers can comprise a blocking group. The first, second, third, and/or fourth primers can lack a fluorophore and/or quencher. Amplicons can be detected using a probe and/or dye.

In some cases, multiple multiplex reactions are carried out, where a first reaction comprises a plurality of pairs of forward primers and reverse primers, wherein each of the forward primers comprises a modified nucleoside at a position corresponding to a different target locus, and each of the forward primers comprises a blocking group. A second multiplex reaction can comprise forward and reverse primers where the modified nucleoside residue of the forward primers match a different allele of the target loci than the forward primers of the first multiplex reaction.

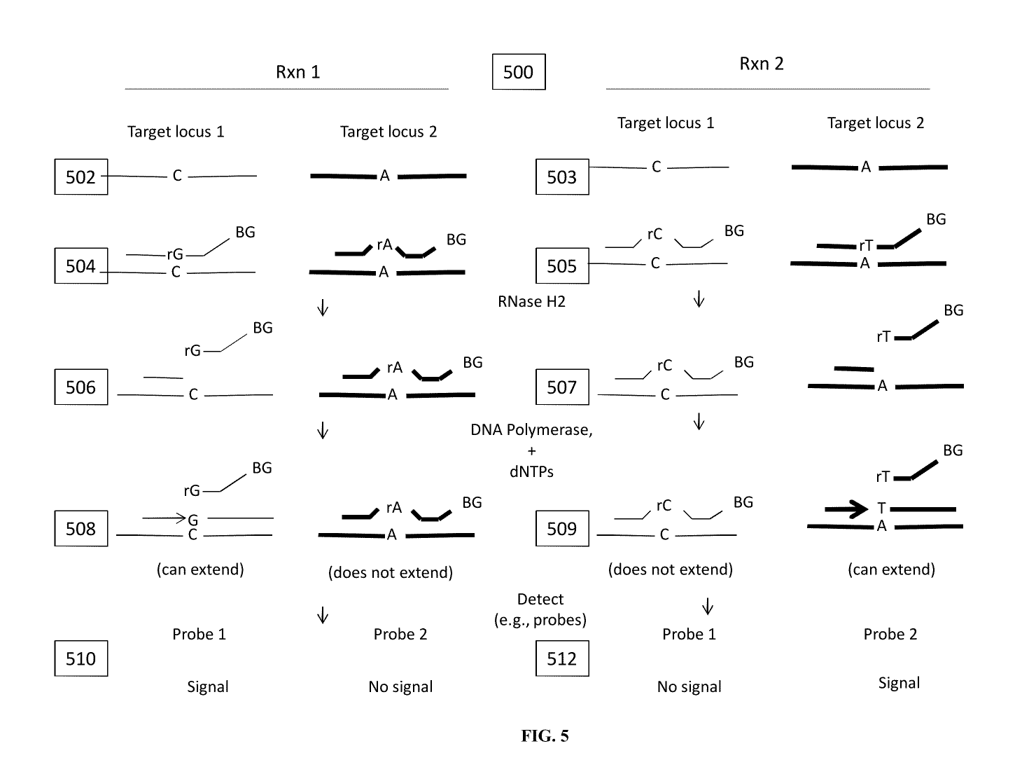

FIG. 5 illustrates an embodiment of multiplexing using forward primers comprising a modified nucleoside at a position corresponding to a target locus and a blocking group, but lacking a fluorophore/quencher pair (500). Two reactions are set up, reaction 1 (rxn 1) and reaction 2 (rxn 2). Rxns 1 and 2 each comprise two target polynucleotides, one comprising target locus 1 and the other comprising target locus 2 (502 and 503). Rxn1 comprises two forward primers: one forward primer comprises a modified nucleoside at a position corresponding to target locus 1 that matches target locus 1, and the other forward primer comprises a modified nucleoside at a position corresponding to target locus 2 that does not match target locus 2 (504). Rxn 2 comprises two forward primers: one forward primer comprises a modified nucleoside at a position corresponding to target locus 1 that does not match target locus 1, and the other forward primer comprises a modified nucleoside at a position corresponding to target locus 2 that does match target locus 2 (505). In Rxn 1, RNase 2 cleaves the primer that matches target locus 1, but does not cleave the primer that does not match target locus 2 (506). In Rxn 2, RNase 2 does not cleave the primer that does not match target locus 1, but does cleave the primer that matches target locus 2 (507). In Rxn 1 and Rxn 2, the cleaved primers are extended while the uncleaved primers are not extended (508 and 509). In Rxn 1, Probe 1 detects amplicons produced from the nucleic acid comprising target locus 1 (510), and in Rxn 2, Probe 2 detects amplicons produced from the nucleic acid comprising target locus 2 (512).

### C2. Blocking Group

In general, a hot-start primer can comprise a blocking group.

A “blocking group” can be a chemical moiety bound to a primer or other oligonucleotide such that an amplification reaction does not occur. For example, primer extension does not occur in the presence of the blocking group. Once the blocking group is removed from the primer, the oligonucleotide is capable of participating in the assay for which it was designed (e.g., PCR). Thus, a “blocking group” can be any chemical moiety that inhibits recognition by a polymerase prevents extension by a polymerase. The blocking group can be incorporated into the cleavage site/domain but can be located on either the 5′- or 3′-side of the cleavage site/domain. The blocking group can be comprised of more than one chemical moiety. A “blocking group” can be removed after hybridization of the oligonucleotide to its target sequence by the cleavage of a digestive enzyme.

A number of blocking groups are known in the art that can be placed at or near the 3′ end of the oligonucleotide (e.g., a primer) to prevent extension. A primer or other oligonucleotide can be modified at the 3′-terminal nucleotide to prevent or inhibit initiation of DNA synthesis by, for example, inverted bases (e.g., inverted dT), the addition of a 3′ deoxyribonucleotide residue (e.g., cordycepin), a 2′,3′-dideoxyribonucleotide residue (e.g., ddC, ddG, ddT, ddA), non-nucleotide linkages or alkane-diol modifications (U.S. Pat. No. 5,554,516). Alkane diol modifications which can be used to inhibit or block primer extension have also been described by Wilk et al., (1990, Nucleic Acids Res., 18 (8):2065), and by Arnold et al., (U.S. Pat. No. 6,031,091). Additional examples of suitable blocking groups include 3′ hydroxyl substitutions (e.g., 3′-phosphate, 3′-triphosphate or 3′-phosphate diesters with alcohols such as 3-hydroxypropyl), a 2′3′-cyclic phosphate, 2′ hydroxyl substitutions of a terminal RNA base (e.g., phosphate or sterically bulky groups such as triisopropyl silyl (TIPS) or tert-butyl dimethyl silyl (TBDMS)). 2′-alkyl silyl groups such as TIPS and TBDMS substituted at the 3′-end of an oligonucleotide are described by Laikhter et al., U.S. patent application Ser. No. 11/686,894 which is incorporated herein by reference. Bulky substituents can also be incorporated on the base of the 3′-terminal residue of the oligonucleotide to block primer extension.

In some cases, the blocking group is photolabile. A photolabile blocking group can include a nitro aromatic compound such as a 2-nitrobenzyl derivative (G. Ciamician and P. Silber, Chem. Ber. 1901, 34, 2040) or an o-nitrobenzyloxy derivative, a benzoin derivative (M. C. Pirrung and L. Fallon, J. Org. Chem. 1998, 63, 241), or benzyl sulfonyl. The nitro aromatic compound may include 6-nitroveratryloxycarbonyl (NVOC), 2-nitrobenzyloxycarbonyl (NBOC), 2-(3,4-methylenedioxy-2-nitrophenyl)oxycarbonyl (MeNPOC), 2-(2-nitrophenyl)propyloxycarbonyl (NPPOC), 2-(2-nitrophenyl)ethylsulfonyl (NPES),2-(2-nitrophenyl)propylsulfonyl (NPPS),2-(3,4-methylenedioxy-2-nitrophenyl)propyloxycarbonyl (MeNPPOC), 2-(5-phenyl-2-nitrophenyl)-propyloxycarbonyl (PbNPPOC), o-nitrobenzylthioethyloxycarbonyl (NBTEOC), o-nitrophenylaminocarbonyl (NPAC), o-nitrophenoxycarbonyl (NPOC), α-methyl-8-nitronaphthylmethoxycarbonyl (MeNMOC), o-nitrophenylthioethyloxycarbonyl, or α,α-dimethyldimethoxybenzyloxycarbonyl (DDZ). In some cases, a photolabile blocking group can be 1-pyrenyl methyloxycarbonyl (PYMOC), anthracenyl-methyloxycarbonyl (ANMOC), dimethoxytritriyl (DMT), and the like, in addition to nitro aromatic compound.

Blocking groups to inhibit primer extension can also be located upstream, that is 5′ from the 3′-terminal residue. Sterically bulky substituents which interfere with binding by the polymerase can be incorporated onto the base, sugar or phosphate group of residues 5′ from the 3′-terminus. Such substituents include bulky alkyl groups like t-butyl, triisopropyl and polyaromatic compounds including fluorophores and quenchers, and can be placed from one to about 10 residues from the 3′-terminus. Alternatively abasic residues such as a C3 spacer can be incorporated in these locations to block primer extension.

In the case of PCR, blocking moieties 5′ of the 3′-terminal residue can serve two functions: 1) to inhibit primer extension, and 2) to block the primer from serving as a template for DNA synthesis when the extension product is copied by synthesis from the reverse primer. The latter is sufficient to block PCR even if primer extension can occur.

A modification used as a blocking group can also be located within a region 3′ to the priming sequence that is non-complementary to the target nucleic acid sequence.

In some cases, the blocking group is a quencher as provided herein. Thus, in these cases a quencher functions as a blocking group as well, and there can be no additional blocking group.

In some cases, a blocking group is a moiety other than a quencher, and as such, a primer comprises only a blocking group without a quencher (e.g., a reverse primer) or comprises a quencher and a non-quencher blocking group (e.g., a forward primer).

In some cases, a forward primer further comprises a blocking residue located 3′ of the position corresponding to the target locus, in additional to the quencher.

In some cases, the blocking residue locates about, less than, more than, or at least 1, 2, 3, 4, 5, 6, 7, 8, 9, 10, 11, 12, 13, 14, 15, 16, 17, 18, 19, 20, 21, 22, 23, 24, or 25 bases from the position corresponding to the target locus.

In some cases, the blocking residue locates between the position corresponding to the target locus and the residue to which the quencher is coupled.

In some cases, the blocking residue locates about, less than, more than, or at least 1, 2, 3, 4, 5, 6, 7, 8, 9, 10, 11, 12, 13, 14, 15, 16, 17, 18, 19, 20, 21, 22, 23, 24, or 25 or more bases from the residue to which the quencher is coupled.

### C3. Primer Labels

In general, primers can be labeled with a fluorophore/quencher pair.

A “fluorescent label” or “fluorophore” can be a compound with a fluorescent emission maximum between about 350 and 900 nm. A wide variety of fluorophores can be used, including but not limited to: 5-FAM (also called 5-carboxyfluorescein; also called Spiro(isobenzofuran-1(3H), 9′-(9H)xanthene)-5-carboxylic acid, 3′,6′-dihydroxy-3-oxo-6-carboxyfluorescein); 5-Hexachloro-Fluorescein; ([4,7,2′,4′,5′,7′-hexachloro-(3′,6′-dipivaloyl-fluoresceinyl)-6-carboxyli-c acid]); 6-Hexachloro-Fluorescein; ([4,7,2′,4′,5′,7′-hexachloro-(3′,6′-dipivaloylfluoresceinyl)-5-carboxylic acid]); 5-Tetrachloro-Fluorescein; ([4,7,2′,7′-tetra-chloro-(3′,6′-dipivaloylfluoresceinyl)-5-carboxylic acid]); 6-Tetrachloro-Fluorescein; ([4,7,2′,7′-tetrachloro-(3′,6′-dipivaloylfluoresceinyl)-6-carboxylic acid]); 5-TAMRA (5-carboxytetramethylrhodamine); Xanthylium, 9-(2,4-dicarboxyphenyl)-3,6-bis(dimethyl-amino); 6-TAMRA (6-carboxytetramethylrhodamine); dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); Cy5 (Indodicarbocyanine-5); Cy3 (Indo-dicarbocyanine-3); and BODIPY FL (2,6-dibromo-4,4-difluoro-5,7-dimethyl-4-bora-3a,4a-diaza-s-indacene-3-pr-oprionic acid); Quasar™-670 dye (Biosearch Technologies); Cal Fluor™ Orange dye (Biosearch Technologies); Rox dyes; Max dyes (Integrated DNA Technologies), as well as suitable derivatives thereof.

A “quencher” can be a molecule or part of a compound, which is capable of reducing the emission from a fluorescent donor when coupled to or in proximity to the donor. Quenching can occur by any of several mechanisms including fluorescence resonance energy transfer, photo-induced electron transfer, paramagnetic enhancement of intersystem crossing, Dexter exchange coupling, and exciton coupling such as the formation of dark complexes. Fluorescence can be “quenched” when the fluorescence emitted by the fluorophore is reduced as compared with the fluorescence in the absence of the quencher by at least 10%, for example, at least 15%, 20%, 25%, 30%, 35%, 40%, 45%, 50%, 55%, 60%, 65%, 70%, 75%, 80%, 85%, 90%, 95%, 98%, 99%, 99.9% or more. The selection of the quencher can depend on the identity of the fluorophore. A number of commercially available quenchers are known in the art, and include but are not limited to DABCYL, Black Hole™ Quenchers (BHQ-1, BHQ-2, and BHQ-3), Iowa Black™ FQ and Iowa Black™ RQ. These are so-called dark quenchers. They have no native fluorescence, virtually eliminating background problems seen with other quenchers such as TAMRA which is intrinsically fluorescent.

Both a fluorophore and a quencher can be coupled to the primer using methods known in the art. In general, a fluorophore can be coupled to the 5′ portion of the hot-start primer and 5′ of the cleavage site. Fluorophores can be added during oligonucleotide synthesis through standard phosphoramidite chemistry. They can also be added post synthesis by introducing a linker with an appropriate functional group during oligo synthesis. Following synthesis, the fluorophore can be coupled to the oligonucleotide functional group. For longer sequences, to permit efficient quenching, the sequence immediately 3′ of the fluorophore and outside the target region of the primer can be made to be partially complementary to permit the formation of a stem-group of a hairpin (i.e. molecular beacon). Thus, the fluorophore can remain with the primer while the primer is hybridized to the target polynucleotide and extended by the polymerase. The quencher can be coupled to the 3′ portion of the hot-start primer and 3′ of the cleavage site. Thus, the quencher can be released from the primer while the primer is hybridized to the target polynucleotide and thus no longer quench the fluorophore that remains coupled to the primer. The proper site of coupling a fluorophore and quencher and the distance between the fluorophore and the quencher can be known in the art. In some cases, a fluorophore is positioned about, more than, less than, or at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases from a quencher in a primer.

### D. Digestive Enzyme

In general, any digestive enzyme that recognizes a match between (hybridization of) a modified residue of the hot-start primer and a given allele at a target locus and cleaves the hot-start primer can be used. In some embodiments, the digestive enzyme is an RNase, such as RNase H enzyme, e.g., RNase H2.

Ribonucleases (RNases) are enzymes that catalyze the hydrolysis of RNA into smaller components. The enzymes are present internally, in bodily fluids, on the surface of skin, and on the surface of many objects, including untreated laboratory glasswear. Double-stranded RNases are present in nearly all intracellular environments and cleave RNA-containing, double-stranded constructs. Single-stranded RNases are ubiquitous in extracellular environments, and are therefore extremely stable in order to function under a wide range of conditions.

The RNase H family is a conserved family of ribonucleases. There are two primary classes of RNase H: RNase H1 and RNase H2. Retroviral RNase H enzymes are similar to the prokaryotic RNase H1. All of these enzymes share the characteristic that they are able to cleave the RNA component of an RNA:DNA heteroduplex. The human and mouse RNase H1 genes are 78% identical at the amino acid level (Cerritelli, et al., (1998) Genomics, 53, 300-307). In prokaryotes, the genes are named rnha (RNase H1) and rnhb (RNase H2). A third family of prokaryotic RNases has been proposed, rnhc (RNase H3) (Ohtani, et al. (1999) Biochemistry, 38, 605-618).

Evolutionarily, “ancient” organisms (archaeal species) in some cases appear to have only a single RNase H enzyme which is most closely related to the modern RNase H2 enzymes (prokaryotic) (Ohtani, et al., J Biosci Bioeng, 88, 12-19). Exceptions do exist, and the archaeal Halobacterium has an RNase H1 ortholog (Ohtani, et al., (2004) Biochem J, 381, 795-802). An RNase H1 gene has also been identified in Thermus thermophilus (Itaya, et al., (1991) Nucleic Acids Res, 19, 4443-4449). RNase H2 enzymes appear to be present in all living organisms. Although all classes of RNase H enzymes hydrolyze the RNA component of an RNA:DNA heteroduplex, the substrate and co-factor requirements can be different. For example, the Type II enzymes can utilize Mg²⁺, Mn²⁺, Co²⁺ (and sometimes Ni²⁺) as cofactor, while the Type I enzymes can require Mg⁺⁺ and can be inhibited by Mn²⁺ ions. The reaction products can be the same for both classes of enzymes: the cleaved products can have a 3′-OH and 5′-phosphate. RNase III class enzymes which cleave RNA:RNA duplexes (e.g., Dicer, Ago2, Drosha) result in similar products and contain a nuclease domain with similarity to RNase H. Most other ribonucleases, and in particular single stranded ribonucleases, result in a cyclic 2′,3′-phosphate and 5′-OH products.

E. coli RNase H1 has been extensively characterized. A large amount of work on this enzyme has been carried out, focusing on characterization of substrate requirements as it impacts antisense oligonucleotide design; this has included studies on both the E. coli RNase H1 (see Crooke, et al., (1995) Biochem J, 312 (Pt 2), 599-608; Lima, et al., (1997) J Biol Chem, 272, 27513-27516; Lima, et al., (1997) Biochemistry, 36, 390-398; Lima, et al., (1997) J Biol Chem, 272, 18191-18199; Lima, et al., (2007) Mol Pharmacol, 71, 83-91; Lima, et al., (2007) Mol Pharmacol, 71, 73-82; Lima, et al., (2003) J Biol Chem, 278, 14906-14912; Lima, et al., (2003) J Biol Chem, 278, 49860-49867) and the Human RNase H1 (see Wu, et al., (1998) Antisense Nucleic Acid Drug Dev, 8, 53-61; Wu, et al., (1999) J Biol Chem, 274, 28270-28278; Wu, et al., (2001) J Biol Chem, 276, 23547-23553). In tissue culture, overexpression of human RNase H1 increases potency of antisense oligos (ASOs) while knockdown of RNase H1 using either siRNAs or ASOs decreases potency of antisense oligonucleotides.

Type I RNase H requires multiple RNA bases in the substrate for full activity. A DNA/RNA/DNA oligonucleotide (hybridized to a DNA oligonucleotide) with only 1 or 2 RNA bases can be inactive. E. coli RNase H1 substrates with three consecutive RNA bases can show weak activity. Full activity can be observed with a stretch of four RNA bases (Hogrefe, et al., (1990) J Biol Chem, 265, 5561-5566). An RNase H1 was cloned from Thermus thermophilus in 1991 which has only 56% amino acid identity with the E. coli enzyme but which has similar catalytic properties (Itaya, et al., (1991) Nucleic Acids Res, 19, 4443-4449). This enzyme was stable at 65° C. but rapidly lost activity when heated to 80° C.

The human RNase H1 gene (Type I RNase H) was cloned in 1998 (Genomics, 53, 300-307 and Antisense Nucleic Acid Drug Dev, 8, 53-61). This enzyme can use a 5 base RNA stretch in DNA/RNA/DNA chimeras for cleavage to occur. Maximal activity was observed in 1 mM Mg²⁺ buffer at neutral pH, and Mn²⁺ ions can be inhibitory (J Biol Chem, 274, 28270-28278). In some cases, cleavage does not occur when 2′-modified nucleosides (such as 2′-OMe, 2′-F, etc.) are substituted for RNA.

The human Type II RNase H was first purified and characterized by Eder and Walder in 1991 (Eder, et al., (1991) J Biol Chem, 266, 6472-6479). This enzyme was initially designated human RNase H1 because it had the characteristic divalent metal ion dependence of what was then known as Class I RNases H. In the current nomenclature, it is a Type II RNase H enzyme. Unlike the Type I enzymes which are active in Mg²⁺ but inhibited by Mn²⁺ ions, the Type II enzymes are active with a wide variety of divalent cations. Optimal activity of human Type II RNase H can observed with 10 mM Mg²⁺, 5 mM Co²⁺, or 0.5 mM Mn²⁺.

Importantly, the substrate specificity of the Type II RNase H (hereafter referred to as RNase H2) is different from RNase H1. In particular, this enzyme can cleave a single ribonucleotide embedded within a DNA sequence (in duplex form) (Eder, et al., (1993) Biochimie, 75, 123-126). Cleavage can occur on the 5′ side of the RNA residue. See review by Kanaya for a summary of prokaryotic RNase H2 enzymes (Kanaya (2001) Methods Enzymol, 341, 377-394).

The E. coli RNase H2 gene has been cloned (Itaya, M. (1990) Proc Natl Acad Sci USA, 87, 8587-8591) and characterized (Ohtani, et al., (2000) J Biochem (Tokyo), 127, 895-899). Like the human enzyme, the E. coli enzyme can functions with Mn²⁺ ions and can be more active with manganese than magnesium.

RNase H2 genes have been cloned and the enzymes characterized from a variety of eukaryotic and prokaryotic sources. The RNase H2 from Pyrococcus kodakaraensis (KOD1) has been cloned and studied in detail (Haruki, et al., (1998) J Bacteriol, 180, 6207-6214; Mukaiyama, et al., (2004) Biochemistry, 43, 13859-13866). The RNase H2 from the related organism Pyrococcus furious has also been cloned but has not been as thoroughly characterized (Sato, et al., (2003) Biochem Biophys Res Commun, 309, 247-252).

The RNase H2 from Methanococcus jannaschii was cloned and characterized by Lai (Lai, et al., (2000) Structure, 8, 897-904; Lai et al., (2003) Biochemistry, 42, 785-791).

In some cases, the hot-start primers utilized in methods and compositions are activated using an RNase H2 enzyme, which can be a thermostable and thermophilic RNase H2 enzyme. Thermostable RNase H2 enzymes and methods for using same are well known in the art (Haruki et al, Gene Cloning and Characterization of Recombinant RNase HII from a Hyperthermophilic Archaeon. Journal of Bacteriology, December 1998, p. 6207-6214.) An exemplary, non-limiting thermostable RNase H2 enzyme is P. abyssi Ribonuclease H2 enzyme. P. abyssi RNaseH2 can be a thermo-stable and thermophilic RNaseH enzyme. The RNaseH enzyme can bind to regions where a ribonucleotide is bound to a deoxyribonucleotide. Once bound, the enzyme can cleave immediately 3′ of the RNA residue. In various cases, the hot start/thermophilic properties of an RNase H2 enzyme used in conjunction with ribo-primers in methods and compositions can be either intrinsic to the enzyme or a result of reversible chemical inactivation or a blocking antibody, as is well known in the art.

In some cases, a detergent is included in the reaction buffer to increase the efficiency of RNase H2. In some more specific cases, the detergent is selected from Triton-X100, Tween-20, and cetyltrimethylammonium bromide (CTAB). The detergent can be a non-ionic detergent. The detergent can be, e.g., NP-40, Brij 52, Brij 58, or Brij 98.

In some cases, hot-start primers contain a reversible chemical modification, for example a 3′ blocking group, that can preclude their suitability to serve as a primer for DNA polymerase, until cleavage by a nuclease enzyme such as RNase H (e.g., RNase H2). In some cases, the nuclease requires correct base pairing in the vicinity of the cleavage site at elevated temperature, in order to become activated. The term “elevated temperatures” in this context refers, in various cases, to a temperature over 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or over 80° C. In some cases, the nuclease exhibits activity at temperatures between about 50 to about 80° C. and does not exhibit appreciable activity or low activity, such as less than 0.1%, 1%, 2%, 5%, 10%, 15%, 20%, 25%, 30%, 35%, 40%, 45%, or 50% of the maximum activity, at temperature such as 37° C., 25° C., 14° C., 4° C., or lower. In some cases, the activity of the enzyme is quantified by looking at the increase in fluorescence signal over time at a given incubation temperature for a preformed substrate. In some cases, the nuclease exhibits activity at the annealing temperature of the reaction and does not exhibit appreciable activity at 25° C. In some cases, the nuclease is thermostable and retains activity (e.g., about 50%, 55%, 60%, 65%, 70%, 75%, 80%, 85%, 90%, 95%, or 99% of the maximum activity) following about, less than, or more than 30, 40, 50, 60, 70, 80, 90-minute incubation at a temperature of 95° C. Each possibility can be considered as being a separate embodiment.

In PCR and qPCR reaction incubations, RNaseH2 can become activated once a high temperature is reached, enabling it to cleave its substrate, thereby liberating the residues 3′ to the RNA residue and the C3 Spacer and enabling amplification. Since RNaseH2 requires that the primer/target match be sufficient to form a strong DNA-RNA duplex in the region near the ribonucleotide, the ribo-primer reaction is usually more specific than a standard oligonucleotide primer. Additionally, since the enzyme can be activated at high temperatures, the ribo-primer reaction can reduce non-specific primer extension, thus reducing undesired nucleotide extension and amplification.

### E. Sample Preparation

A target polynucleotide can be prepared from various samples of interest.

### E1. Tissue Acquisition and Preparation

The methods and compositions of the present disclosure provide methods for obtaining genetic material, such as fetal or maternal genetic material, or a biopsy sample of a subject having a cancer or being suspect of having a cancer. Often, the fetal/maternal genetic material is obtained via a blood draw, or other method provided herein. In some cases, the starting material is maternal plasma or peripheral blood, such as maternal peripheral venous blood. The peripheral blood cells can be enriched for a particular cell type (e.g., mononuclear cells; red blood cells; CD4+ cells; CD8+ cells; B cells; T cells, NK cells, or the like). The peripheral blood cells can also be selectively depleted of a particular cell type (e.g., mononuclear cells; red blood cells; CD4+ cells; CD8+ cells; B cells; T cells, NK cells, or the like). The starting material can also be bone marrow-derived mononuclear cells. The starting material can also include tissue extracted directly from a placenta (e.g., placental cells) or umbilical cord (e.g., umbilical vein endothelial cells, umbilical artery smooth muscle cell, umbilical cord blood cells). The starting material can also derive directly from the fetus in the form, e.g., of fetal tissue, e.g., fetal fibroblasts or blood cells. The starting material can also be from an infant or child, including neonatal tissue.

This starting material can be obtained in some cases from a hospital, laboratory, clinical or medical laboratory. In some cases, a sample can be derived from a non-cellular entity comprising nucleic acid (e.g., a virus) or from a cell-based organism (e.g., member of archaea, bacteria, or eukarya domains). The sample can be from a plant, fungi, eubacteria, archeabacteria, protist, or animal. The animal can be a fish, e.g., a zebrafish. The animal can be a mammal. The mammal can be, e.g., a dog, cat, horse, cow, mouse, rat, or pig. The mammal can be a primate, e.g., a human, chimpanzee, orangutan, or gorilla. The human can be a male or female. The sample can be from a human embryo or human fetus. The human can be an infant, child, teenager, adult, or elderly person. A sample can be obtained from an animal that is pregnant. The pregnant animal can be a human. In some cases, the sample is taken from a subject (e.g., an expectant mother) at at least 4, 5, 6, 7, 8, 9, 10, 11, 12, 13, 14, 15, 16, 17, 18, 19, 20, 21, 22, 23, 24, 25, or 26 weeks of gestation. In some cases, the subject is affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs). In other cases, the sample is taken from a female patient of child-bearing age and, in some cases, the female patient is not pregnant or of unknown pregnancy status. In still other cases, the subject is a male patient, a male expectant father, or a male patient at risk of, diagnosed with, or having a specific genetic abnormality. In some cases, the female patient is known to be affected by, or is a carrier of, a genetic disease or genetic variation, or is at risk of, diagnosed with, or has a specific genetic abnormality. In some cases, the status of the female patient with respect to a genetic disease or genetic variation can not be known. In further cases, the sample is taken from any child or adult patient of known or unknown status with respect to copy number variation of a genetic sequence. In some cases, the child or adult patient is known to be affected by, or is a carrier of, a genetic disease or genetic variation.

In some cases, in order to obtain sufficient nucleic acid for testing, a blood volume of at least 1, 2, 3, 4, 5, 10, 20, 25, 30, 35, 40, 45, or 50 mL is drawn. This blood volume can provide at least 1,000 genome equivalents (GE) of total DNA. Total DNA is present at roughly 1,000 GE/mL of maternal plasma in early pregnancy, and a fetal DNA concentration of about 3.5% of total plasma DNA. However, less blood can be drawn for a genetic screen where less statistical significance is required, or the DNA sample is enriched for fetal DNA. Also, the fetal DNA concentration can vary according to the gestational age of the fetus. In some cases, fetal DNA or RNA can be enriched by isolating red blood cells, in particular fetal nucleated red blood cells, which differ from anucleated adult red blood cells, as described below. In other cases, red blood cells can be removed from a maternal blood sample, and genetic material can be obtained from maternal plasma.

In some cases, the starting material can be a tissue sample comprising a solid tissue, with non-limiting examples including brain, liver, lung, kidney, prostate, ovary, spleen, lymph node (including tonsil), thyroid, pancreas, heart, skeletal muscle, intestine, larynx, esophagus, and stomach. In other cases, the starting material can be cells containing nucleic acids, including connective tissue, muscle tissue, nervous tissue, and epithelial cells, and in particular exposed epithelial cells such as skin cells and hair cells. In yet other cases, the starting material can be a sample containing nucleic acids, from any organism, from which genetic material can be obtained and detected by droplet digital PCR, as outlined herein.

In some cases, the sample is a biopsy. A biopsy can be preserved by formalin-fixing or formalin-fixing and paraffin-embedding (FFPE) a tissue obtained in a biopsy. A biopsy can be processed into smaller pieces. In some cases, the biopsy can be treated to preserve RNA, e.g., with RNAlater® or RNASafer™. In some cases, the sample comprises one or more reagents that inhibit RNases. The biopsy can be stored on wet ice (approximately 4° C.), at room temperature (approximately 25° C.), at approximately −20° C., at approximately −80° C., e.g., stored on dry ice, in liquid nitrogen or a dry ice/alcohol slurry.

If the tissue is frozen, the tissue can be frozen within about 0.5, 1, 5, 10, 15, 30, 60, 120, 150, 180, 210, or 240 minutes of surgical resection. Fixative agents that can be used on the biopsy tissue include, e.g., methanol-acetone, ethanol, acetone, formalin, Carnoy's fixative (60% ethanol, 30% chloroform, 10% glacial acetic acid), Bouin's fixative, methacarn (substitute 60% methanol for the ethanol in Carnoy), FINEfix, Omnifix, and UMFIX (universal molecular fixative).

### E2. Extraction of DNA or RNA

Genomic DNA can be isolated from plasma (e.g., maternal plasma) using techniques known in the art, such as using the Qiagen Midi Kit for purification of DNA from blood cells. DNA can be eluted in 100 □l of distilled water. The Qiagen Midi Kit can also be used to isolate DNA from the maternal cells contained in the buffy coat. A QIAamp Circulating Nucleic Acid Kit can also be used for such purposes, see, e.g., www.qiagen.com/products/qiaampcirculatingnucleicacidkit.aspx.

Methods of extracting polynucleotides (e.g., DNA) can also include the use of liquid extraction (e.g, Trizol, DNAzol) techniques.

For example, the starting sample (e.g., blood or plasma) can have a starting volume of 15-30 ml, from which about 100-200 ul of DNA or other polynucleotide can be extracted. The 200 ul of DNA of the extracted sample can then be converted (or concentrated) into a final sample with a smaller volume, e.g., 5 ul, 10 ul. In some cases, the volume of the starting sample can be greater than 2-, 5-, 10-, 20-, 30-, 40-, 50-, 75-, 100-, 500-, 1000-, 5000-, 10,000-, 50,000-, 100,000-, 500,000-, or 1,000,000-fold the volume of the final sample. The final sample can also be a sample that is introduced into a device for droplet generation.

The final sample can be from about 1 to about 20 ul in volume. In some cases, the final sample is greater than 1, 5, 10, 15, 20, 25, 30, 35, 40, 45, 50, 75, or 100 ul. In some cases, the final sample is less than 1, 5, 10, 15, 20, 25, 30, 35, 40, 45, 50, 75, or 100 ul. In some cases, the final sample is greater than 1, 5, 10, 15, 20, 25, 30, 35, 40, 45, 50, 75, or 100 nl. In some cases, the final sample is less than 1, 5, 10, 15, 20, 25, 30, 35, 40, 45, 50, 75, or 100 nl. In some cases, the final sample is greater than 1, 5, 10, 15, 20, 25, 30, 35, 40, 45, 50, 75, or 100 pl. In some cases, the final sample is less than 1, 5, 10, 15, 20, 25, 30, 35, 40, 45, 50, 75, or 100 pl.

In some cases, DNA can be concentrated by known methods, including centrifugation and the use of various enzyme inhibitors (e.g. for DNase). The DNA can be bound to a selective membrane (e.g., silica) to separate it from contaminants. The DNA can also be enriched for fragments circulating in the plasma which are less than 1000, 500, 400, 300, 200 or 100 base pairs in length. This size selection can be done on a DNA size separation medium, such as an electrophoretic gel or chromatography material (Huber et al. (1993) Nucleic Acids Res. 21:1061-6), gel filtration chromatography, TSK gel (Kato et al. (1984) J. Biochem, 95:83-86). In some cases, the polynucleotide (e.g., DNA, RNA) can be selectively precipitated, concentrated (e.g., sample can be subjected to evaporation), or selectively captured using a solid-phase medium. Following precipitation, DNA or other polynucleotide can be reconstituted or dissolved into a small volume. A small volume can enable hybridization, or enable improved hybridization, of a probe with a target polynucleotide.

In some cases, the starting material can comprise cells or tissue, including connective tissue, muscle tissue, nervous tissue, blood cells, or epithelial cells. In some cases, non-nucleic acid materials can be removed from the starting material using enzymatic treatments (such as protease digestion). Other non-nucleic acid materials can be removed in some cases by treatment with membrane-disrupting detergents and/or lysis methods (e.g., sonication, French press, freeze/thaw, dounce), which can be followed by centrifugation to separate nucleic acid-containing fractions from non-nucleic acid-containing fractions. The extracted nucleic acid can be from any appropriate sample including but not limited to, nucleic acid-containing samples of tissue, bodily fluid (for example, blood, serum, plasma, saliva, urine, tears, peritoneal fluid, ascitic fluid, vaginal secretion, breast fluid, breast milk, lymph fluid, cerebrospinal fluid or mucosa secretion), umbilical cord blood, chorionic villi, amniotic fluid, an embryo, a two-celled embryo, a four-celled embryo, an eight-celled embryo, a 16-celled embryo, a 32-celled embryo, a 64-celled embryo, a 128-celled embryo, a 256-celled embryo, a 512-celled embryo, a 1024-celled embryo, embryonic tissues, lymph fluid, cerebrospinal fluid, mucosa secretion, or other body exudate, fecal matter, an individual cell or extract of the such sources that contain the nucleic acid of the same, and subcellular structures such as mitochondria, using protocols well established within the art.

In one embodiment, blood can be collected into an apparatus containing a magnesium chelator including but not limited to EDTA, and is stored at 4° C. Optionally, a calcium chelator, including but not limited to EGTA, can be added. A chelator can be, e.g., EDTA, EGTA, citric acid, L-(+)-tartaric acid, potassium oxalate, sodium citrate, sodium L-tartrate, ammonium citrate, potassium tetraoxalate, ethylene glycol-bis(2-aminoethylether)-N,N,N′,N′-tetraacetic acid, potassium citrate, potassium oxalate, sodium bitartrate, 5-sulfosalicylic acid, or nitrilotriacetic acid. In another embodiment, a cell lysis inhibitor is added to the maternal blood including but not limited to formaldehyde, formaldehyde derivatives, formalin, glutaraldehyde, glutaral-dehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, or cleavable crosslinkers. These agents can be added to non-blood samples.

Plasma RNA extraction is described, e.g., in Enders et al. (2003), Clinical Chemistry 49:727-731. Briefly, plasma harvested after centrifugation steps can be mixed with Trizol LS reagent (Invitrogen) and chloroform. The mixture can be centrifuged, and the aqueous layer transferred to new tubes. Ethanol can be added to the aqueous layer. The mixture can then be applied to an RNeasy mini column (Qiagen) and processed according to the manufacturer's recommendations.

In some cases when the extracted material comprises single-stranded RNA, double-stranded RNA, or DNA-RNA hybrid, these molecules can be converted to double-stranded DNA using techniques known in the field. For example, reverse transcriptase can be employed to synthesize DNA from RNA molecules. In some cases, conversion of RNA to DNA can require a prior ligation step, to ligate a linker fragment to the RNA, thereby permitting use of universal primers to initiate reverse transcription. In other cases, the poly-A tail of an mRNA molecule, for example, can be used to initiate reverse transcription. Following conversion to DNA, the methods detailed herein can be used, in some cases, to further capture, select, tag, or isolate a desired sequence.

In some cases, fetal RNA found in maternal blood (as well as RNA in general) can be analyzed as well. As described previously, “mRNA of placental origin is readily detectable in maternal plasma,” (Ng et al. (2003) Proc. Nat. Acad. Sci. 100:4748-4753), hPL (human placental lactogen) and hCG (human chorionic gonadotropin) mRNA transcripts are detectable in maternal plasma, as analyzed using the respective real-time RT-PCR assays. In the present method, mRNA encoding genes expressed in the placenta and present on a chromosome of interest can be used. For example, DSCR4 (Down syndrome critical region 4) is found on chromosome 21 and is mainly expressed in the placenta. Its mRNA sequence can be found at GenBank NM_{—}005867. In this case, RNase H minus (RNase ^{H-}) reverse transcriptases (RTs) can be used to prepare cDNA for detection. RNase ^{H-} RTs are available from several manufacturers, such as SuperScript™ II (Invitrogen). Reverse transcriptase PCR can be used as described herein for chromosomal DNA. The RNA can include siRNA, miRNA, cRNA, tRNA, rRNA, mRNA, or any other type of RNA. RNA molecules can include non-coding RNA such as siRNA, micro RNA, piwi-interacting RNA (piRNA), small nuclear ribonucleic acid (sn RNA), small hairpin RNA (short hairpin RNA; shRNA), subgenomic mRNA, spliced RNA, unspliced RNA, exosomal RNA, as well viral genome RNA. The RNA can be cell-free RNA derived from a blood sample. RNA can include spliced RNA and unspliced RNA. In some cases, the RNA is synthetic.

The sample containing the genetic target can comprise genomic DNA in the form of whole chromosomes, chromosomal fragments, or non-chromosomal fragments. In some cases, the average length of the genomic DNA fragment can be less than 100, 200, 300, 400, 500, or 800 base pairs, or less than 1, 2, 5, 10, 20, 30, 40, 50, 60, 70, 80, 90, 100, 110, 120, 130, 140, 150, 160, 170, 180, 190, or 200 nucleotides, or less than 1, 2, 5, 10, 20, 30, 40, 50, 60, 70, 80, 90, 100 kilobases. In some cases, the fragments range from about 10 to about 500, about 10 to about 1000, or about 100 to about 150 bases (or nucleotides) in length, and, in some cases, between about 100 to about 150 bases.

The methods described herein can be used to amplify a target sequence from a relative short template, such as a sample containing a template that is about, more than, less than, or at least 40, 45, 50, 55, 60, 65, 70, 75, 80, 85, 90, 95, or 100 bases.

### F. RT Reaction

In some cases, the target polynucleotides are prepared from a RNA using RT-PCR.

In yet another embodiment, the methods described herein can be used in coupled reverse transcription-PCR (RT-PCR). In one such embodiment reverse transcription and PCR are carried out in two distinct steps. First, a cDNA copy of the sample mRNA can be synthesized using either an oligo dT primer or a sequence specific primer. Random hexamers and the like can also be used to prime cDNA synthesis. The resulting cDNA can be then used as the substrate for PCR employing the blocked primers and methods described herein.

Alternatively reverse transcription and PCR can be carried out in a single closed tube reaction. In one such embodiment three primers are employed, one for reverse transcription and two for PCR. The primer for reverse transcription can bind to the mRNA 3′ to the position of the PCR amplicon. Although not essential, reverse transcription primer can include RNA residues or modified analogs such as 2′-O-methyl RNA bases which will not form a substrate for RNase H when hybridized to the mRNA. An RNase H2 enzyme which has decreased activity at lower temperatures can be used as the cleaving agent.

In the three primer RT-PCR assay it can be desirable to inhibit the RT-primer from participating in the PCR reaction. This inhibition can be accomplished by utilizing an RT-primer having a lower Tm than the PCR primers so it will not hybridize under the PCR conditions. Alternatively, a non-replicable primer incorporating, for example, two adjacent C3 spacers can be used as the RT-primer (as in polynomial amplification, see U.S. Pat. No. 7,112,406). In this case when the cDNA is copied by extension of the forward PCR primer it will not include the binding site for the RT-primer.

In one embodiment, only the reverse PCR primer is blocked utilizing the compositions and methods described herein. In yet another embodiment both the forward and reverse PCR primers are blocked. The reverse PCR primer can be blocked in the 3 primer RT-PCR assay to prevent it from being utilized for reverse transcription. If desired, modified bases such as 2′-O-methyl RNA residues can be incorporated in the reverse PCR primer although any such modification can allow the primer sequence to serve as a template for DNA synthesis and be copied.

In some cases of the two primer RT-PCR assays described herein, only the forward PCR is blocked. In some cases, the reverse PCR primer also serves as the RT-primer and therefore cannot be blocked.

The temperature to carry out the RT reaction can depend on the reverse transcriptase being used. In some cases, a thermostable reverse transcriptase is used and the RT reaction is carried out at about 55° C. to about 75° C., about 55° C. to about 60° C., or about 60° C. In some cases, the RT reaction is carried out at about, more than, or at least 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

The RT reaction and the PCR reaction can be carried out in various formats known in the art, such as in tubes, microtiter plates, microfluidic devices, or droplets.

RT reaction can be carried out in standard recommended volumes ranging from about 5 μL to about 100 μL, or from about 10 μL to about 20 μL reaction volumes. In droplets, reaction volumes range from about 1 pL to about 100 nL, or about 10 pL to about 1 nL. In some cases, the RT reaction is carried out in a droplet having a volume that is about or less than 1 nL.

In some cases, the PCR reaction is in a droplet having a reaction volume ranging from about 1 pL to about 100 nL, or about 10 pL to about 1 nL. In some cases, the PCR reaction is carried out in a droplet having a volume that is about or less than about 1 nL.

In some cases, the RT reaction and the PCR reaction are carried out in a same droplet having a reaction volume ranging from about 1 pL to about 100 nL, or about 10 pL to about 1 nL. In some cases, an RT reaction and a PCR reaction are carried out in a droplet having a volume that is about or less than 1 nL, or a volume that is about or less than 1 pL.

In some cases, the RT reaction and the PCR reaction are carried out in a plurality of droplets each having a reaction volume ranges from about 1 pL to about 100 nL, or about 10 pL to about 1 nL. In some cases, RT reaction and the PCR reaction are carried out in a droplet having a volume that is about or less than 1 nL.

### G. PCR Reaction/Amplification

In another aspect, provided herein is a PCR reaction mixture comprising: a) a set of primers, comprising at least one forward primer and at least one reverse primer, that is capable of amplifying a target polynucleotide sequence characteristic of a species of interest (hereinafter referred to as a “species-specific gene”); and b) a digestive enzyme, wherein at least one of said forward primer and said reverse primer is provided as hot-start primers or a mixture of hot-start primers and non-hot-start primers. Typically, the reaction mixture further comprises one or more of a DNA polymerase enzyme, deoxynucleoside triphosphates (dNTPs), and a divalent cation, most often a magnesium ion. The hot-start primers contain an inactivating chemical modification that is reversed by the action of the digestive enzyme present in the reaction mixture. Each individual hot-start primer molecule becomes a substrate for the digestive enzyme when the hot-start primer is hybridized to a sequence complementary to the primer at elevated temperatures.

Techniques for amplification of target and reference sequences are known in the art and include the methods described in U.S. Pat. No. 7,041,481. Briefly, the techniques include methods and compositions that separate samples into small droplets, in some instances with each containing on average less than one nucleic acid molecule (polynucleotide) per droplet, amplifying the nucleic acid sequence in each droplet and detecting the presence of a target nucleic acid sequence.

A PCR reaction can be carried out using a DNA polymerase known in the art. The DNA polymerase can be a DNA-dependent DNA polymerase.

Any DNA polymerase that catalyzes primer extension can be used including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™. Genomic DNA polymerase, or sequenase. In some cases, a thermostable DNA polymerase is used. A hot start PCR can also be performed wherein the reaction is heated to 95° C. for two minutes prior to addition of the polymerase or the polymerase can be kept inactive until the first heating step in cycle 1. Hot start PCR can be used to minimize nonspecific amplification. Any number of PCR cycles can be used to amplify the DNA, e.g., about, more than about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 cycles. The number of amplification cycles can be about 1 to about 45, about 10 to about 45, about 20 to about 45, about 30 to about 45, about 35 to about 45, about 10 to about 40, about 10 to about 30, about 10 to about 25, about 10 to about 20, about 10 to about 15, about 20 to about 35, about 25 to about 35, about 30 to about 35, or about 35 to about 40.

In some cases, the use of Taqman probe for detection is obviated, which can require the presence of an enzyme possessing 5′-nuclease activity. Any thermophilic DNA polymerase can be used for amplification. In some cases, a DNA polymerase used herein substantially lacks 5′-nuclease activity and/or 3′-5′ exonuclease activity (e.g., proof-reading DNA polymerases).

Amplification of target nucleic acids can be performed by any means known in the art. Target nucleic acids can be amplified by polymerase chain reaction (PCR) or isothermal DNA amplification. Examples of PCR techniques that can be used include, but are not limited to, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR, digital PCR, droplet digital PCR, and emulsion PCR. Other suitable amplification methods include the ligase chain reaction (LCR), transcription amplification, molecular inversion probe (MIP) PCR, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938, as well as include Q beta replicase mediated RNA amplification. Amplification of target nucleic acids can occur on a bead. In other cases, amplification does not occur on a bead. Amplification can be isothermal amplification, e.g., isothermal linear amplification.

Thermocycling reactions can be performed on samples contained in droplets. Droplets can be polydisperse or monodisperse, generated through agitation, sonication or microfluidically through a T-channel junction or other means by those familiar with the art. Densities can exceed about 20,000 droplets/40 ul (1 nl droplets), or about 200,000 droplets/40 ul (100 pL droplets). The droplets can remain intact during thermocycling. Droplets can remain intact during thermocycling at densities of about, at least, less than, or greater than 10,000 droplets/mL, 100,000 droplets/mL, 200,000 droplets/mL, 300,000 droplets/mL, 400,000 droplets/mL, 500,000 droplets/mL, 600,000 droplets/mL, 700,000 droplets/mL, 800,000 droplets/mL, 900,000 droplets/mL, 1,000,000 droplets/mL, 5 million droplets/mL, 10 million droplets/mL, 25 million droplets/mL, 50 million droplets/mL, 100 million droplets/mL, 250 million droplets/mL, 500 million droplets/mL, 750 million droplets/mL, 1 billion droplets/mL, 2.5 billion droplets/mL, 5 billion droplets/mL, 7.5 billion droplets/mL, or 10 billion droplets/mL. In some cases, droplet density is about 10,000 droplets/mL to about 100,000 droplets/mL, about 100,000 droplets/mL to about 1 million droplets/mL, about 1 million droplets/mL to about 10 million droplets/mL, about 10 million droplets/mL to about 100 million droplets/mL, about 100 million droplets/mL to about 1 billion droplets/mL, or about 1 billion droplets/mL to about 10 billion droplets/mL. In other cases, two or more droplets can coalesce during thermocycling. In other cases, greater than 100 or greater than 1,000 droplets can coalesce during thermocycling.

In some cases, one or more probes are used to detect amplicons. For example, probes can be used to detect amplicons when primers lack fluorophore/quencher pairs. A probe can be selected to ensure that it does not bind a target locus in an assay, or to other non-target polynucleotides in a sample. A label (Fluorophore, dye) used on a probe (e.g., a Taqman probe) to detect a target nucleic acid sequence or reference nucleic acid sequence in the methods described herein can be, e.g., 6-carboxyfluorescein (FAM), tetrachlorofluorescin (TET), 4,7,2′-trichloro-7′-phenyl-6-carboxyfluorescein (VIC), HEX, Cy3, Cy 3.5, Cy 5, Cy 5.5, Cy 7, tetramethylrhodamine, ROX, and JOE. The label can be an Alexa Fluor dye, e.g., Alexa Fluor 350, 405, 430, 488, 532, 546, 555, 568, 594, 633, 647, 660, 680, 700, and 750. The label can be Cascade Blue, Marina Blue, Oregon Green 500, Oregon Green 514, Oregon Green 488, Oregon Green 488-X, Pacific Blue, Rhodamine Green, Rhodol Green, Rhodamine Green-X, Rhodamine Red-X, and Texas Red-X. A probe can be a hydrolysis probe, e.g., a 5′ hydrolysis probe; a Taqman probe can be a 5′ hydrolysis probe.

In some cases, a probe comprises nucleotides that are complementary to one another such that the probe can form a hairpin, with about 18 to about 30 bases complementary to a target polynucleotide, e.g., 3′ of a target locus in a target polynucleotide. The stem of the hairpin can comprise about 5 to about 7 nucleotides on each side of the hairpin that can base-pair to one another. The 5′ end can comprise a fluorophore, and the 3′ end can comprise a quencher. In some cases, the 5′ end of the probe comprises a quencher, and the 3′ end comprises a fluorophore. Because of the stem, a fluorophore and quencher at the ends of the probe can be in close proximity. When the probe anneals to a target polynucleotide, the stem can denature, resulting in the fluorophore and quencher being separated and an increase in fluorescence signal. In some cases, the fluorophore and/or quencher are not located at the 5′ and/or 3′ ends of a probe.

Fluorescent probes (such as molecular beacons or TaqMan probes) can be used to light up partitions that contain amplified template molecules. The number of illuminated partitions can provide a direct measure of the number of target molecules in the sample.

In some cases, a primer/probe is used; in some cases, the primer/probe is a Scorpion™ probe. A Scorpion™ probe can provide a FRET-based stem-loop detection mechanism similar to Molecular Beacon, except that the probe also has a segment attached that serves as an amplification primer (see e.g., Whitcombe et al. Nat. Biotechnol. 1999, August 17(8): 804-7; U.S. Pat. No. 6,326,145). A Scorpion™ probe can maintain a stem-loop configuration in the unhybridized state with the fluorophore thereby quenched. A Scorpion™ probe can have a longer multi-component structure, e.g., a 5′ fluorophore, then a target-specific stem-loop section, then a quencher (e.g., Black Hole Quencher™), then a blocker (e.g., hexethelene glycol (HEG)), and finally a 3′ primer sequence. The blocker can prevent reverse extension of the product onto the probe. After primer extension occurs, the Scorpion™ probe can be attached to the terminal end of the amplicon. When denaturation occurs again, followed by annealing, the loop segment of the probe can preferentially bind to its long complementary segment on the attached template, thereby opening the stem-loop structure and releasing fluorescence. Alternatively, the stem-loop structure can be cut into two units with one unit having four components, e.g., a 5′ fluorophore, a target specific segment, a blocker and a primer, and the other unit having the quencher and a probe segment.

In some cases, a primer/probe is a Sunrise™ probe. A Sunrise™ probe can comprise a primer attached to a hairpin probe that is extended during amplification. This arrangement can separate the internal quencher label from the 5′ terminal fluorophore (Nazarenko et al., Nucl. Acids Res. 1997, 25: 2516-2521).

In some cases, a probe comprises a 5′ modification to prevent incorporation of the probe into an amplicon.

In some cases, a reaction mixture comprises about, more than, less than, or at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 150, 200, 250, 300, 350, 400, 450, or 500 probes.

In another aspect, the methods provided herein comprise detecting the amplicons generated by the amplification, e.g., by detecting the fluorescent emission of the fluorophore coupled to the forward primers.

Fluorescence detection can be achieved using a variety of detector devices equipped with a module to generate excitation light that can be absorbed by a fluorescence reader, as well as a module to detect light emitted by the fluorescence reader. In some cases, samples (such as droplets) can be detected in bulk. For example, samples can be allocated in plastic tubes that are placed in a detector that measures bulk fluorescence from plastic tubes. In some cases, one or more samples (such as droplets) can be partitioned into one or more wells of a plate, such as a 96-well or 384-well plate, and fluorescence of individual wells can be detected using a fluorescence plate reader or a real-time PCR machine.

In some cases, the detection of amplicons is carried out in real-time. The methods provided herein can be suitable for any type of PCR reaction, either quantitative (“real-time”) or non-quantitative. In certain cases, methods provided herein are particularly suitable for threshold value amplification reactions, of which real-time PCR is provided as a non-limiting exemplary embodiment. “Threshold value amplification reaction” refers to a nucleic acid amplification reaction wherein the assay determines a threshold value such as for example the cycle number at which amplification of a particular target sequence above a threshold level is achieved. In one non-limiting embodiment, fluorescence emission from the unquenched fluorophores are utilized to monitor amplification.

qPCR is well known in the art. qPCR reaction mixtures can comprise the 4 naturally-occurring deoxynucleoside triphosphates (dNTPs); a divalent cation, and a polymerase enzyme. The divalent cation can be a magnesium ion. In some cases, a thermophilic DNA polymerase enzyme is utilized. qPCR has been used for detecting human and animal target nucleic acid sequences and sequences specific to pathogens in a variety of applications.

In some cases, the detector further comprises handling capabilities for droplet samples, with individual droplets entering the detector, undergoing detection, and then exiting the detector. For example, a flow cytometry device can be adapted for use in detecting fluorescence from droplet samples. In some cases, a microfluidic device equipped with pumps to control droplet movement is used to detect fluorescence from droplets in single file. In some cases, droplets are arrayed on a two-dimensional surface and a detector moves relative to the surface, detecting fluorescence at each position containing a single droplet.

Following acquisition of fluorescence detection data, a computer can be used to store and process the data. A computer-executable logic can be employed to perform such functions as subtraction of background fluorescence, assignment of target and/or reference sequences, and quantification of the data. A computer can be useful for displaying, storing, retrieving, or calculating diagnostic results from the molecular profiling; displaying, storing, retrieving, or calculating raw data from genomic or nucleic acid expression analysis; or displaying, storing, retrieving, or calculating any sample or patient information useful in the methods provided herein.

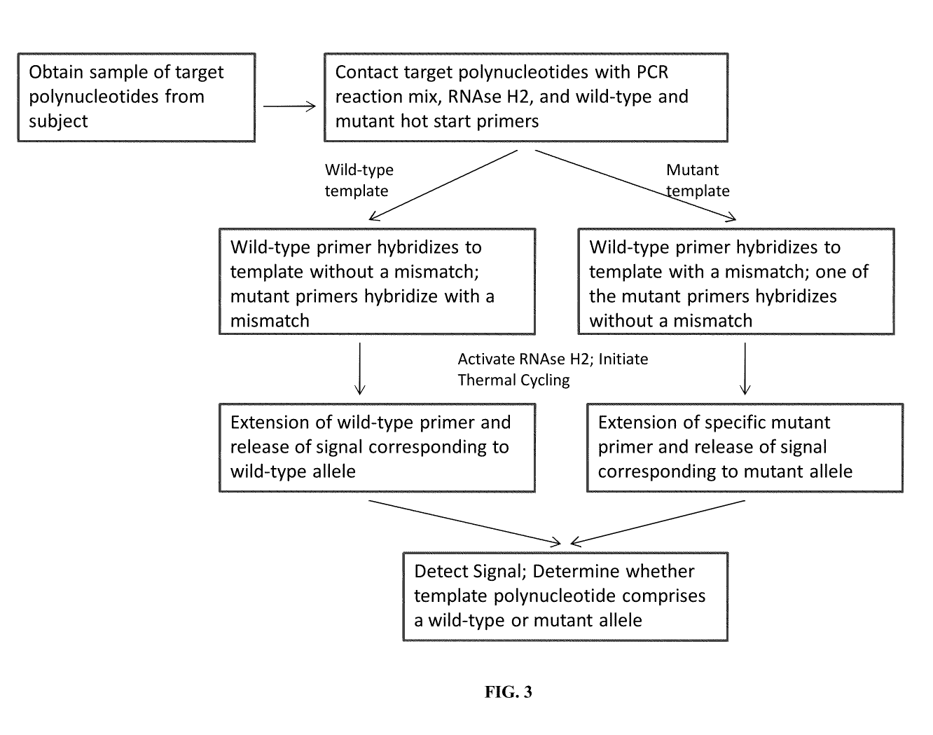

An exemplary work-flow is depicted in FIG. 3. In general, a sample comprising a target polynucleotide is obtained from subject such as human. Then the target polynucleotide is in contact with a PCR reaction mix, together with RNAse H2, and wild-type and mutant hot start primers. If a wild-type template is present, the wild-type primer hybridizes to template without a mismatch and mutant primers hybridize with a mismatch. If a mutant template is present, the wild-type primer hybridizes to template with a mismatch and one of the mutant primers hybridizes without a mismatch. The reaction is then brought to a higher temperature to activate RNAse H2, which cleaves the perfectly matched hot-start-primer and template to initiate the primer extension. Then thermal cycling is carried out. In some cases, RNaseH2 cleavage is concomitant with the PCR, and there is no pre-incubation with RNase H2. Extension of wild-type primer and release of signal corresponding to wild-type allele, and extension of specific mutant primer and release of signal corresponding to mutant allele. The signal is detected to determine whether template polynucleotide comprises a wild-type or mutant allele. The detection can be carried in real-time or as an end-point assay (such as in digital PCR). The PCR reaction can also be carried out either in bulk or digitally (such as in ddPCR).

### H. Droplet Generation

In some cases, an RT reaction and/or DNA amplification reaction (e.g. PCR) are carried out in droplets, such as in droplet digital PCR. The droplets used herein could include emulsion compositions (or mixtures of two or more immiscible fluids) as described in U.S. Pat. No. 7,622,280. The droplets could be generated by devices described in WO/2010/036352. The term emulsion, as used herein, can refer to a mixture of immiscible liquids (such as oil and water). Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets can be oil droplets within a continuous aqueous phase. The droplets provided herein are designed to prevent mixing between compartments, with each compartment protecting its contents from evaporation and coalescing with the contents of other compartments.

The mixtures or emulsions described herein can be stable or unstable. The emulsions can be relatively stable and have minimal coalescence. Coalescence occurs when small droplets combine to form progressively larger ones. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets generated from a droplet generator coalesce with other droplets. The emulsions can also have limited flocculation, a process by which the dispersed phase comes out of suspension in flakes.

Splitting a sample into small reaction volumes as described herein can enable the use of reduced amounts of reagents, thereby lowering the material cost of the analysis. Reducing sample complexity by partitioning also improves the dynamic range of detection because higher-abundance molecules are separated from low-abundance molecules in different compartments, thereby allowing lower-abundance molecules greater proportional access to reaction reagents, which in turn enhances the detection of lower-abundance molecules.

Droplets can be generated having an average diameter of about, less than, at least, or more than 0.001, 0.01, 0.05, 0.1, 1, 5, 10, 20, 30, 40, 50, 60, 70, 80, 100, 120, 130, 140, 150, 160, 180, 200, 300, 400, or 500 microns. Droplets can have an average diameter of about 0.001 to about 500, about 0.01 to about 500, about 0.1 to about 500, about 0.1 to about 100, about 0.01 to about 100, or about 1 to about 100 microns. Microfluidic methods of producing emulsion droplets using microchannel cross-flow focusing or physical agitation are known to produce either monodisperse or polydisperse emulsions. The droplets can be monodisperse droplets. The droplets can be generated such that the size of the droplets does not vary by more than plus or minus 5% of the average size of the droplets. In some cases, the droplets are generated such that the size of the droplets does not vary by more than plus or minus 2% of the average size of the droplets. A droplet generator can generate a population of droplets from a single sample, wherein none of the droplets vary in size by more than plus or minus about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

Higher mechanical stability can be useful for microfluidic manipulations and higher-shear fluidic processing (e.g., in microfluidic capillaries or through 90 degree turns, such as valves, in fluidic path). Pre- and post-thermally treated droplets or capsules can be mechanically stable to standard pipet manipulations and centrifugation.

A droplet can be formed by flowing an oil phase through an aqueous sample. The aqueous phase can comprise a buffered solution and reagents for performing a PCR reaction, including nucleotides, primers, probe(s) for fluorescent detection, template nucleic acids, DNA polymerase enzyme, and optionally, reverse transcriptase enzyme.

The aqueous phase can comprise a buffered solution and reagents for performing a PCR reaction without solid-state beads, such as magnetic-beads. The buffered solution can comprise about, at least, more than, or less than 1, 5, 10, 15, 20, 30, 50, 100, or 200 mM Tris. In some cases, the concentration of potassium chloride can be about, at least, more than, or less than 10, 20, 30, 40, 50, 60, 80, 100, 200 mM. The buffered solution can comprise about 15 mM Tris and about 50 mM KCl. The nucleotides can comprise deoxyribonucleotide triphosphate molecules, including dATP, dCTP, dGTP, dTTP, in concentrations of about, at least, more than, or less than 50, 100, 200, 300, 400, 500, 600, or 700 μM each. In some cases dUTP is added within the aqueous phase to a concentration of about, at least, more than, or less than 50, 100, 200, 300, 400, 500, 600, or 700, 800, 900, or 1000 μM. In some cases, magnesium chloride or magnesium acetate (MgCl₂) is added to the aqueous phase at a concentration of about, at least, more than, or less than 1.0, 2.0, 3.0, 4.0, or 5.0 mM. The concentration of MgCl₂ can be about 3.2 mM. In some cases, magnesium acetate or magnesium is used. In some cases, magnesium sulfate is used.

A non-specific blocking agent such as BSA or gelatin from bovine skin can be used, wherein the gelatin or BSA is present in a concentration range of about 0.1 to about 0.9% w/v. Other possible blocking agents can include betalactoglobulin, casein, dry milk, or other common blocking agents. In some cases, concentrations of BSA and gelatin are about 0.1% w/v.

Primers for amplification within the aqueous phase can have a concentration of about, at least, more than, or less than 0.05, 0.1, 0.2, 0.3, 0.4, 0.5, 0.6, 0.7, 0.8, 0.9, 1.0, 1.2, 1.5, 1.7, or 2.0 μM. Primer concentration within the aqueous phase can be about 0.05 to about 2, about 0.1 to about 1.0, about 0.2 to about 1.0, about 0.3 to about 1.0, about 0.4 to about 1.0, or about 0.5 to about 1.0 μM. The concentration of primers can be about 0.5 μM. The aqueous phase can comprise one or more probes for fluorescent detection, at a concentration of about, at least, more than, or less than 0.05, 0.1, 0.2, 0.3, 0.4, 0.5, 0.6, 0.7, 0.8, 0.9, 1.0, 1.2, 1.4, 1.6, 1.8, or 2.0 μM. The aqueous phase can comprise one or more probes for fluorescent detection, at a concentration of about 0.05 to about 2.0, about 0.1 to about 2.0, about 0.25 to about 2.0, about 0.5 to about 2.0, about 0.05 to about 1, about 0.1 to about 1, or about 0.1 to about 0.5 μM. The concentration of probes for fluorescent detection can be about 0.25 μM. Amenable ranges for target nucleic acid concentrations in PCR are between about 1 pg and about 500 ng.

In some cases, the aqueous phase can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors).

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, Zonyl FSN. Pluronic F-68 can be present at a concentration of about 0.5% w/v.

In some cases magnesium sulfate can be substituted for magnesium chloride, at similar concentrations. A wide range of common, commercial PCR buffers from varied vendors can be substituted for the buffered solution.

The oil phase can comprise a fluorinated base oil which can be additionally stabilized by combination with a fluorinated surfactant such as a perfluorinated polyether. In some cases, the base oil can be one or more of HFE 7500, FC-40, FC-43, FC-70, or other common fluorinated oil. In some cases, the anionic surfactant is Ammonium Krytox (Krytox-AM), the ammonium salt of Krytox FSH, or morpholino derivative of Krytox-FSH. Krytox-AS can be present at a concentration of about, more than about, or less than about 0.1%, 0.2%, 0.3%, 0.4%, 0.5%, 0.6%, 0.7%, 0.8%, 0.9%, 1.0%, 2.0%, 3.0%, or 4.0% w/w. In some cases, the concentration of Krytox-AS is 1.8%. In other cases, the concentration of Krytox-AS is 1.62%. Morpholino derivative of Krytox-FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% w/w. The concentration of morpholino derivative of Krytox-FSH can be about 1.8%. The concentration of morpholino derivative of Krytox-FSH can be about 1.62%.

The oil phase can further comprise an additive for tuning the oil properties, such as vapor pressure or viscosity or surface tension. Nonlimiting examples include perfluoro-octanol and 1H,1H,2H,2H-Perfluorodecanol. 1H,1H,2H,2H-Perfluorodecanol can be added to a concentration of about, more than about, or less than about 0.05%, 0.06%, 0.07%, 0.08%, 0.09%, 1.00%, 1.25%, 1.50%, 1.75%, 2.00%, 2.25%, 2.50%, 2.75%, or 3.00% w/w. 1H,1H,2H,2H-Perfluorodecanol can be added to a concentration of about 0.18% w/w.

The emulsion can formulated to produce highly monodisperse droplets having a liquid-like interfacial film that can be converted by heating into microcapsules having a solid-like interfacial film; such microcapsules can behave as bioreactors able to retain their contents through a reaction process such as PCR amplification. The conversion to microcapsule form can occur upon heating. For example, such conversion can occur at a temperature of greater than about 50, 60, 70, 80, 90, or 95 degrees Celsius. In some cases this heating occurs using a thermocycler. During the heating process, a fluid or mineral oil overlay can be used to prevent evaporation. In some cases, excess continuous phase oil is removed prior to heating. In some cases, excess continuous phase oil is not removed prior to heating. The biocompatible capsules can be resistant to coalescence and/or flocculation across a wide range of thermal and mechanical processing.

Following conversion, the capsules can be stored at about, at least, more than, or less than 3, 4, 5, 6, 7, 8, 9, 10, 15, 20, 25, 30, 35, or 40 degrees. These capsules can be useful in biomedical applications, such as stable, digitized encapsulation of macromolecules, particularly aqueous biological fluids containing a mix of nucleic acids or protein, or both together; drug and vaccine delivery; biomolecular libraries; clinical imaging applications, and others.

The microcapsules can contain one or more polynucleotides and can resist coalescence, particularly at high temperatures. Accordingly, PCR amplification reactions can occur at a very high density (e.g., number of reactions per unit volume). In some cases, greater than 100,000, 500,000, 1,000,000, 1,500,000, 2,000,000, 2,500,000, 5,000,000, or 10,000,000 separate reactions can occur per ml. In some cases, the reactions occur in a single well, e.g., a well of a microtiter plate, without inter-mixing between reaction volumes. The microcapsules can also contain other components necessary to enable a PCR reaction to occur, e.g., primers, probes, dNTPs, DNA or RNA polymerases, etc. These capsules exhibit resistance to coalescence and flocculation across a wide range of thermal and mechanical processing.

In some cases, the droplet is generated using commercially available droplet generator, such as Bio-Rad QX100™ Droplet Generator. RT and the droplet PCR is carried out using commercially available, and the droplet is analyzed using commercially available droplet reader such as generator, such as Bio-Rad QX100™ Droplet Reader.

In some cases, the amplifying step is carried out by performing digital PCR, such as microfluidic-based digital PCR or droplet digital PCR.

In some cases, the digital PCR is performed in droplets having a volume that is between about 1 pL and about 100 nL.

In some cases, droplet generation comprise encapsulating dyes, such as fluorescent molecules, in droplets, for example, with a known concentration of dyes, where the droplets are suspended in an immiscible carrier fluid, such as oil, to form an emulsion. Exemplary fluorescent dyes that can used with the present system include a fluorescein derivative, such as carboxyfluorescein (FAM), and a PULSAR 650 dye (a derivative of Ru(bpy)₃). FAM has a relatively small Stokes shift, while Pulsar® 650 dye has a very large Stokes shift. Both FAM and PULSAR 650 dye can be excited with light of approximately 460-480 nm. FAM emits light with a maximum of about 520 nm (and not substantially at 650 nm), while PULSAR 650 dye emits light with a maximum of about 650 nm (and not substantially at 520 nm). Carboxyfluorescein can be paired in a probe with, for example, BLACK HOLE Quencher™ 1 dye, and PULSAR 650 dye can be paired in a probe with, for example, BLACK HOLE Quencher™ 2 dye. For example, fluorescent dyes include, but are not limited to, DAPI, 5-FAM, 6-FAM, 5(6)-FAM, 5-ROX, 6-ROX, 5,6-ROX, 5-TAMRA, 6-TAMRA, 5(6)-TAMRA SYBR, TET, JOE, VIC, HEX, R6G, Cy3, NED, Cy3.5, Texas Red, Cy5, and Cy5.5.

### I. Digital Analysis

The methods provided herein are suitable for use with a digital analysis technique. The digital analysis can be digital polymerase chain reaction (digital PCR, DigitalPCR, dPCR, or dePCR). The dPCR can be droplet dPCR (ddPCR).

In some cases, the methods comprise using droplet dPCR (ddPCR) where an extreme high level of enhancement in sensitivity is achieved by leveraging the removal of background template through partitioning with the inherent sensitivity provided by the hot-start primer amplification system provided herein. For example, in bulk PCR reactions, the sensitivity is about 1/100 to 1/10,000, inclusive, or e.g., 1/100 to 1/1,000, as defined by mutant/(mutant+wild-type). Using ddPCR, this sensitivity is manifest in each partition, such as across 20,000 droplets, the sensitivity is about 1/1,000 to 1/100,000, inclusive.

In general, dPCR can involve spatially isolating (or partitioning) individual polynucleotides from a sample and carrying out a polymerase chain reaction on each partition. The partition can be, e.g., a well (e.g., wells of a microwell plate), capillary, dispersed phase of an emulsion, a chamber (e.g, a chamber in an array of miniaturized chambers), a droplet, or a nucleic acid binding surface. The sample can be distributed so that each partition has 0 or 1 polynucleotides. After PCR amplification, the number of partitions with or without a PCR product can be enumerated. The total number of partitions can be about, at least, or more than 500, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 150,000, 200,000, 500,000, 750,000, 1,000,000, 2,500,000, 5,000,000, 7,500,000, 10,000,000, 25,000,000, 50,000,000, 75,000,000, or 100,000,000. In some cases, the total number of partitions is about 1000 to about 10,000, about 10,000 to about 100,000, about 100,000 to about 1,000,000, about 1,000,000 to about 10,000,000, or about 10,000,000 to about 100,000,000. Positive and negative droplets can be counted.

In some cases, less than 0.00001, 0.00005, 0.00010, 0.00050, 0.001, 0.005, 0.01, 0.05, 0.1, 0.5, 1, 2, 2.5, 3, 3.5, 4, 4.5, 5, 6, 7, 8, 9, or 10 copies of target polynucleotide can detected. In some cases, less than about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 150, 200, 250, 300, 350, 400, 450, or 500 copies of a target polynucleotide are detected. In some cases, the droplets described herein are generated at a rate of greater than 1, 2, 3, 4, 5, 10, 50, 100, 200, 300, 400, 500, 600, 700, 800, 900, 1000, 2500, 5000, 10,000, 25,000, 50,000, 75,000, 100,000, 250,000, 500,000, or 1,000,000 droplets/second. In some cases, the droplets described herein are generated at a rate of about 1 to about 10, about 10 to about 100, about 100 to about 1000, about 1000 to about 10,000, about 10,000 to about 100,000, or about 100,000 to about 1,000,000 droplets/second.

An integrated, rapid, flow-through thermal cycler device can be used in the methods described herein. See, e.g., International Application No. PCT/US2009/005317, filed 9-23-2009. In such an integrated device, a capillary is wound around a cylinder that maintains 2, 3, or 4 temperature zones. As droplets flow through the capillary, they are subjected to different temperature zones to achieve thermal cycling. The small volume of each droplet results in an extremely fast temperature transition as the droplet enters each temperature zone.

A digital PCR device (e.g., droplet digital PCR device) for use with the methods, compositions, and kits described herein can detect multiple signals (see e.g. U.S. Provisional Patent Application No. 61/454,373, filed Mar. 18, 2011, herein incorporated by reference in its entirety).

### II. Kits

Also provided herein are kits for nucleic acid amplification and detection that allow for use of the primers and other novel oligonucleotides in the aforementioned methods. In some cases, the kits include a container containing a cleavage compound, for example a nicking enzyme or an RNase H enzyme; another container containing a DNA polymerase and an instruction booklet for using the kits. In certain cases, the kits include a container containing an RNase H enzyme combined with a DNA polymerase. Optionally, the modified oligonucleotides used in the assay can be included with the enzymes. The cleavage enzyme agent, DNA polymerase and oligonucleotides used in the assay can be stored in a state where they exhibit long-term stability, e.g., in suitable storage buffers or in a lyophilized or freeze dried state. In addition, the kits can further comprise a buffer for the nicking agent or RNase H, a buffer for the DNA polymerase or DNA ligase, or both buffers. Alternatively, the kits can further comprise a buffer suitable for both RNase H and the DNA polymerase. Buffers can include RNasin and other inhibitors of single stranded ribonucleases. Descriptions of various components of the present kits can be found in preceding sections related to various methods described herein.

In some cases, a kit comprises a primer that is complementary to a first sequence of a target polynucleotide, comprising: (a) a first modified nucleoside residue at a position corresponding to a target locus residing within said first sequence; (b) a fluorophore coupled to said primer and is 5′ of said position corresponding to the target locus; and (c) a quencher coupled to said first primer and is 3′ of said position corresponding to the target locus.

Optionally, a kit can contain an instruction booklet providing information on how to use the kit for amplifying nucleic acids in the presence of the novel primers and/or other novel oligonucleotides. In certain cases, the information includes one or more descriptions on how to use and/or store the RNase H, DNA polymerase, and oligonucleotides used in the assay as well as descriptions of buffer(s) for the RNase H and the DNA polymerase, appropriate reaction temperature(s) and reaction time period(s), etc.

Accordingly, in one embodiment, a kit for the selective amplification of a nucleic acid from a sample is provided. The kit comprises (a) a first and a second oligonucleotide primer, each having a 3′ end and 5′ end, wherein each primer is complementary to a portion of a nucleic acid to be amplified or its complement, and wherein at least one primer comprises (i) a first modified nucleoside residue at a position corresponding to a target locus residing within said first sequence; (ii) a fluorophore coupled to said primer and 5′ of said position corresponding to the target locus; and (iii) a quencher coupled to said first primer and 3′ of said position corresponding to the target locus; and optionally a blocking group linked at or near to the 3′ end of the oligonucleotide to prevent primer extension and/or to prevent the primer from being copied by DNA synthesis directed from the opposite primer; (b) an RNase H enzyme; and (c) an instruction manual for amplifying the nucleic acid. The kit can optionally include a DNA polymerase.

In a further embodiment, the kit for selective amplification of a nucleic acid includes an oligonucleotide probe having a 3′ end and a 5′ end comprising an RNase H cleavable domain, a fluorophore and a quencher, wherein the cleavable domain is positioned between the fluorophore and the quencher, and wherein the probe is complementary to a portion of the nucleic acid to be amplified or its complement.

Also provided herein are reaction mixtures including a set of primers for amplifying a particular species-specific gene, wherein the primers for amplifying at least one end of the target sequence include hot-start primers, and optionally non-hot-start primers.

### III. Applications

The PCR provided herein can be used in any applications known in the art that requires the determination of allele identity in a sample, which is useful in diagnosis and drug development.

One specific embodiment provided herein is a method of detecting an antibiotic-resistant strain of a pathogen in a clinical specimen, the method comprising the steps of: (a) performing real-time PCR on nucleic acids from the test sample, wherein the PCR reaction mixture is a reaction mixture described herein; (b) determining the Ct values of the signals generated by the probes that detect a pathogen-specific sequence, whether a gene or intergenic region (herein referred to as a “pathogen-specific gene”), and a polynucleotide sequence that confers antibiotic resistance (herein referred to as an “antibiotic resistance gene”); and (c) comparing the Ct value of the pathogenic-specific gene to the Ct value of the antibiotic resistance gene. In another embodiment, the method further comprises the steps of amplifying a “bridging region” (a region connecting the usual point of insertion of an element containing an antibiotic resistance gene [the “insertion point”] and a known location in the genome of the target pathogen) and determining the Ct value of the bridging region. In another embodiment, provided herein is a kit for detecting an antibiotic-resistant strain in a test sample. Each possibility can be considered as being a separate embodiment.

In some cases, the compositions, methods, or kits provided herein are used for the detection of specific point mutations associated with the onset and progression of cancers. Compositions and methods described herein can also be applied to acquired mutations during the course of therapeutic intervention in the treatment of cancers that can decide the course and efficacy of a given treatment. It can also be used to measure the efficacy of a treatment, i.e. minimal residual disease. Minimal residual disease (MRD) refers to small numbers of leukemic cells that remain in the patient during treatment, or after treatment when the patient is in remission (no symptoms or signs of disease). It can be the major cause of relapse in cancer and leukemia. In some cases, the methods and compositions provided herein are used to measure minute levels of cancer cells in tissue samples, such as low as one cancer cell in a million normal cells.

In some cases, the compositions, methods, or kits provided herein can be used to analyze pharmacokinetics or pharmacodynamics. Different patients can react to a drug differently based on the SNP profiles of the patients.

Conditions

The methods, compositions, and kits described herein can be used to detect, diagnose, prognose, and/or monitor one or more cancers or conditions. The condition or cancer can include, for example, acute myeloid leukemia; bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, limited (1-3) metastatic lesions, multiple (>3) metastatic lesions, carcinomatous lymphomatous meningitis, nonimmunosuppressed primary CNS lymphoma, and metastatic spine tumors; cervical cancer; chronic myelogenous leukemia (CML); colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type 1, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T-Cell lymphoma, and mycosis fungoides/Sezary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hiirthle cell neoplasm, medullary carcinoma, and anaplastic carcinoma; uterine neoplasms, including endometrial cancer and uterine sarcoma.

Means of studying cancer are described, e.g., in U.S. Pat. No. 6,869,760 and U.S. Patent Application Publication No. 20070178461.

The methods, compositions, and kits described herein can be used to detect, diagnose, prognose, or monitor an autoimmune disease. The autoimmune disease can be, e.g., systemic lupus erythematosus, multiple sclerosis, rheumatoid arthritis, or Ankylosing Spondylitis. Methods of investigating autoimmune diseases are described, e.g., in U.S. Pat. Nos. 5,641,864 and 6,617,171.

In some cases, the methods, compositions, and kits described herein can be used to detect, diagnose, prognose, or monitor a neurological or neurocognitive condition. The neurological or neurocognitive condition can be a neurological disorder listed on the National Institute of Neurological Disorders and Stroke webpage (www.ninds.nih gov/disorders/disorder_index.htm). In some cases, the subject can have a sign or symptom. The neurological or neurocognitive condition, can be, e.g., abarognosis (e.g., loss of the ability to detect the weight of an object held in the hand or to discern the difference in weight between two objects), acid lipase disease, acid maltase deficiency, acquired epileptiform aphasia, absence of the septum pellucidum, acute disseminated encephalomyelitis, adie's pupil, Adie's syndrome, adrenoleukodystrophy, agenesis of the corpus callosum, agnosia, Aicardi syndrome, Aicardi-Goutieres syndrome disorder, AIDS—neurological complications, akathisia, alcohol related disorders, Alexander disease, Alien hand syndrome (anarchic hand), allochiria, Alpers' disease, altitude sickness, alternating hemiplegia, Alzheimer's disease, amyotrophic lateral sclerosis, anencephaly, aneurysm, Angelman syndrome, angiomatosis, anoxia, Antiphospholipid syndrome, aphasia, apraxia, arachnoid cysts, arachnoiditis, arnold-chiari malformation, Asperger syndrome, arteriovenous malformation, ataxia, ataxias and cerebellar or spinocerebellar degeneration, ataxia telangiectasia, atrial fibrillation, stroke, attention deficit hyperactivity disorder, auditory processing disorder, autism, autonomic dysfunction, back pain, Barth syndrome, Batten disease, becker's myotonia, Behcet's disease, bell's palsy, benign essential blepharospasm, benign focal amyotrophy, benign intracranial hypertension, Bernhardt-Roth syndrome, bilateral frontoparietal polymicrogyria, Binswanger's disease, blepharospasm, Bloch-Sulzberger syndrome, brachial plexus birth injuries, brachial plexus injury, Bradbury-Eggleston syndrome, brain or spinal tumor, brain abscess, brain aneurysm, brain damage, brain injury, brain tumor, Brown-Séquard syndrome, bulbospinal muscular atrophy, CADASIL (cerebral autosomal dominant arteriopathy subcortical infarcts and leukoencephalopathy), Canavan disease, Carpal tunnel syndrome, causalgia, cavernomas, cavernous angioma, cavernous malformation, Central cervical cord Syndrome, Central cord syndrome, Central pain syndrome, central pontine myelinolysis, centronuclear myopathy, cephalic disorder, ceramidase deficiency, cerebellar degeneration, cerebellar hypoplasia, cerebral aneurysm, cerebral arteriosclerosis, cerebral atrophy, cerebral beriberi, cerebral cavernous malformation, cerebral gigantism, cerebral hypoxia, cerebral palsy, cerebral vasculitis, Cerebro-Oculo-Facio-Skeletal syndrome (COFS), cervical spinal stenosis, Charcot-Marie-Tooth disease, chiari malformation, Cholesterol ester storage disease, chorea, choreoacanthocytosis, Chronic fatigue syndrome, chronic inflammatory demyelinating polyneuropathy (CIDP), chronic orthostatic intolerance, chronic pain, Cockayne syndrome type II, Coffin-Lowry syndrome, colpocephaly, coma, Complex regional pain syndrome, compression neuropathy, concussion, congenital facial diplegia, congenital myasthenia, congenital myopathy, congenital vascular cavernous malformations, corticobasal degeneration, cranial arteritis, craniosynostosis, cree encephalitis, Creutzfeldt-Jakob disease, cumulative trauma disorders, Cushing's syndrome, Cytomegalic inclusion body disease (CIBD), cytomegalovirus infection, Dancing eyes-dancing feet syndrome (opsoclonus myoclonus syndrome), Dandy-Walker syndrome (DWS), Dawson disease, decompression sickness, De morsier's syndrome, dejerine-klumpke palsy, Dejerine-Sottas disease, Delayed sleep phase syndrome, dementia, dementia—multi-infarct, dementia—semantic, dementia—subcortical, dementia with lewy bodies, dentate cerebellar ataxia, dentatorubral atrophy, depression, dermatomyositis, developmental dyspraxia, Devic's syndrome, diabetes, diabetic neuropathy, diffuse sclerosis, Dravet syndrome, dysautonomia, dyscalculia, dysgraphia, dyslexia, dysphagia, dyspraxia, dyssynergia cerebellaris myoclonica, dyssynergia cerebellaris progressiva, dystonia, dystonias, Early infantile epileptic, Empty sella syndrome, encephalitis, encephalitis lethargica, encephalocele, encephalopathy, encephalopathy (familial infantile), encephalotrigeminal angiomatosis, encopresis, epilepsy, epileptic hemiplegia, erb's palsy, erb-duchenne and dejerine-klumpke palsies, erythromelalgia, essential tremor, extrapontine myelinolysis, Fabry's disease, Fahr's syndrome, fainting, familial dysautonomia, familial hemangioma, familial idiopathic basal ganglia calcification, familial periodic paralyses, familial spastic paralysis, Farber's disease, febrile seizures, fibromuscular dysplasia, fibromyalgia, Fisher syndrome, floppy infant syndrome, foot drop, Foville's syndrome, friedreich's ataxia, frontotemporal dementia, Gaucher's disease, generalized gangliosidoses, Gerstmann's syndrome, Gerstmann-Straussler-Scheinker disease, giant axonal neuropathy, giant cell arteritis, Giant cell inclusion disease, globoid cell leukodystrophy, glossopharyngeal neuralgia, Glycogen storage disease, gray matter heterotopia, Guillain-Barré syndrome, Hallervorden-Spatz disease, head injury, headache, hemicrania continua, hemifacial spasm, hemiplegia alterans, hereditary neuropathies, hereditary spastic paraplegia, heredopathia atactica polyneuritiformis, herpes zoster, herpes zoster oticus, Hirayama syndrome, Holmes-Adie syndrome, holoprosencephaly, HTLV-1 associated myelopathy, HIV infection, Hughes syndrome, Huntington's disease, hydranencephaly, hydrocephalus, hydrocephalus—normal pressure, hydromyelia, hypercortisolism, hypersomnia, hypertension, hypertonia, hypotonia, hypoxia, immune-mediated encephalomyelitis, inclusion body myositis, incontinentia pigmenti, infantile hypotonia, infantile neuroaxonal dystrophy, Infantile phytanic acid storage disease, Infantile refsum disease, infantile spasms, inflammatory myopathy, inflammatory myopathies, iniencephaly, intestinal lipodystrophy, intracranial cyst, intracranial hypertension, Isaac's syndrome, Joubert syndrome, Karak syndrome, Kearns-Sayre syndrome, Kennedy disease, Kinsbourne syndrome, Kleine-Levin syndrome, Klippel feil syndrome, Klippel-Trenaunay syndrome (KTS), Klüiver-Bucy syndrome, Korsakoffs amnesic syndrome, Krabbe disease, Kugelberg-Welander disease, kuru, Lafora disease, lambert-eaton myasthenic syndrome, Landau-Kleffner syndrome, lateral femoral cutaneous nerve entrapment, Lateral medullary (wallenberg) syndrome, learning disabilities, Leigh's disease, Lennox-Gastaut syndrome, Lesch-Nyhan syndrome, leukodystrophy, Levine-Critchley syndrome, lewy body dementia, Lipid storage diseases, lipoid proteinosis, lissencephaly, Locked-In syndrome, Lou Gehrig's, lumbar disc disease, lumbar spinal stenosis, lupus—neurological sequelae, lyme disease—neurological sequelae, Machado-Joseph disease (spinocerebellar ataxia type 3), macrencephaly, macropsia, megalencephaly, Melkersson-Rosenthal syndrome, Menieres disease, meningitis, meningitis and encephalitis, Menkes disease, meralgia paresthetica, metachromatic leukodystrophy, metabolic disorders, microcephaly, micropsia, migraine, Miller fisher syndrome, mini-stroke (transient ischemic attack), misophonia, mitochondrial myopathy, Mobius syndrome, Moebius syndrome, monomelic amyotrophy, mood disorder, Motor neurone disease, motor skills disorder, Moyamoya disease, mucolipidoses, mucopolysaccharidoses, multi-infarct dementia, multifocal motor neuropathy, multiple sclerosis, multiple system atrophy, multiple system atrophy with orthostatic hypotension, muscular dystrophy, myalgic encephalomyelitis, myasthenia—congenital, myasthenia gravis, myelinoclastic diffuse sclerosis, myoclonic encephalopathy of infants, myoclonus, myopathy, myopathy—congenital, myopathy—thyrotoxic, myotonia, myotonia congenita, myotubular myopathy, narcolepsy, neuroacanthocytosis, neurodegeneration with brain iron accumulation, neurofibromatosis, Neuroleptic malignant syndrome, neurological complications of AIDS, neurological complications of lyme disease, neurological consequences of cytomegalovirus infection, neurological manifestations of AIDS, neurological manifestations of pompe disease, neurological sequelae of lupus, neuromyelitis optica, neuromyotonia, neuronal ceroid lipofuscinosis, neuronal migration disorders, neuropathy—hereditary, neurosarcoidosis, neurosyphilis, neurotoxicity, neurotoxic insult, nevus cavernosus, Niemann-pick disease, Non 24-hour sleep-wake syndrome, nonverbal learning disorder, normal pressure hydrocephalus, O'Sullivan-McLeod syndrome, occipital neuralgia, occult spinal dysraphism sequence, Ohtahara syndrome, olivopontocerebellar atrophy, opsoclonus myoclonus, Opsoclonus myoclonus syndrome, optic neuritis, orthostatic hypotension, Overuse syndrome, chronic pain, palinopsia, panic disorder, pantothenate kinase-associated neurodegeneration, paramyotonia congenita, Paraneoplastic diseases, paresthesia, Parkinson's disease, paroxysmal attacks, paroxysmal choreoathetosis, paroxysmal hemicrania, Parry-Romberg syndrome, Pelizaeus-Merzbacher disease, Pena shokeir II syndrome, perineural cysts, periodic paralyses, peripheral neuropathy, periventricular leukomalacia, persistent vegetative state, pervasive developmental disorders, photic sneeze reflex, Phytanic acid storage disease, Pick's disease, pinched nerve, Piriformis syndrome, pituitary tumors, PMG, polio, polymicrogyria, polymyositis, Pompe disease, porencephaly, Post-polio syndrome, postherpetic neuralgia (PHN), postinfectious encephalomyelitis, postural hypotension, Postural orthostatic tachycardia syndrome, Postural tachycardia syndrome, Prader-Willi syndrome, primary dentatum atrophy, primary lateral sclerosis, primary progressive aphasia, Prion diseases, progressive hemifacial atrophy, progressive locomotor ataxia, progressive multifocal leukoencephalopathy, progressive sclerosing poliodystrophy, progressive supranuclear palsy, prosopagnosia, Pseudo-Torch syndrome, Pseudotoxoplasmosis syndrome, pseudotumor cerebri, Rabies, Ramsay hunt syndrome type I, Ramsay hunt syndrome type II, Ramsay hunt syndrome type III, Rasmussen's encephalitis, Reflex neurovascular dystrophy, Reflex sympathetic dystrophy syndrome, Refsum disease, Refsum disease—infantile, repetitive motion disorders, repetitive stress injury, Restless legs syndrome, retrovirus-associated myelopathy, Rett syndrome, Reye's syndrome, rheumatic encephalitis, rhythmic movement disorder, Riley-Day syndrome, Romberg syndrome, sacral nerve root cysts, saint vitus dance, Salivary gland disease, Sandhoff disease, Schilder's disease, schizencephaly, schizophrenia, Seitelberger disease, seizure disorder, semantic dementia, sensory integration dysfunction, septo-optic dysplasia, severe myoclonic epilepsy of infancy (SMEI), Shaken baby syndrome, shingles, Shy-Drager syndrome, Sjögren's syndrome, sleep apnea, sleeping sickness, snatiation, Sotos syndrome, spasticity, spina bifida, spinal cord infarction, spinal cord injury, spinal cord tumors, spinal muscular atrophy, spinocerebellar ataxia, spinocerebellar atrophy, spinocerebellar degeneration, Steele-Richardson-Olszewski syndrome, Stiff-Person syndrome, striatonigral degeneration, stroke, Sturge-Weber syndrome, subacute sclerosing panencephalitis, subcortical arteriosclerotic encephalopathy, SUNCT headache, superficial siderosis, swallowing disorders, sydenham's chorea, syncope, synesthesia, syphilitic spinal sclerosis, syringohydromyelia, syringomyelia, systemic lupus erythematosus, tabes dorsalis, tardive dyskinesia, tardive dysphrenia, tarlov cyst, Tarsal tunnel syndrome, Tay-Sachs disease, temporal arteritis, tetanus, Tethered spinal cord syndrome, Thomsen disease, thomsen's myotonia, Thoracic outlet syndrome, thyrotoxic myopathy, tic douloureux, todd's paralysis, Tourette syndrome, toxic encephalopathy, transient ischemic attack, transmissible spongiform encephalopathies, transverse myelitis, traumatic brain injury, tremor, trigeminal neuralgia, tropical spastic paraparesis, Troyer syndrome, trypanosomiasis, tuberous sclerosis, ubisiosis, uremia, vascular erectile tumor, vasculitis syndromes of the central and peripheral nervous systems, viliuisk encephalomyelitis (VE), Von economo's disease, Von Hippel-Lindau disease (VHL), Von recklinghausen's disease, Wallenberg's syndrome, Werdnig-Hoffman disease, Wernicke-Korsakoff syndrome, West syndrome, Whiplash, Whipple's disease, Williams syndrome, Wilson's disease, Wolman's disease, X-linked spinal and bulbar muscular atrophy, or Zellweger syndrome.

Methods of investigating neurological conditions are described, e.g., in U.S. Patent Application Publication No. 20120207726 and 2011018390.

The methods, compositions, and kits provided herein can be used to detect or monitor cells or viruses potentially associated with a bioterrorist or biowarfare attack. In some cases, the methods, compositions, and kits described herein can be used for forensics or epidemiology. The methods, compositions, and kits described herein can be used to monitor an epidemic or a pandemic, e.g., an influenza pandemic. In some cases, the methods, compositions, and kits described herein can be used in archaeology.

Mutations/Copy Number Variations

The methods described herein can be used to detect copy number variations. Diseases associated with copy number variations can include, for example, DiGeorge/velocardiofacial syndrome (22q11.2 deletion), Prader-Willi syndrome (15q11-q13 deletion), Williams-Beuren syndrome (7q11.23 deletion), Miller-Dieker syndrome (MDLS) (17p13.3 microdeletion), Smith-Magenis syndrome (SMS) (17p11.2 microdeletion), Neurofibromatosis Type 1 (NF1) (17q11.2 microdeletion), Phelan-McErmid Syndrome (22q13 deletion), Rett syndrome (loss-of-function mutations in MECp2 on chromosome Xq28), Merzbacher disease (CNV of PLP1), spinal muscular atrophy (SMA) (homozygous absence of telomerec SMN1 on chromosome 5q13), Potocki-Lupski Syndrome (PTLS, duplication of chromosome 17p. 11.2). Additional copies of the PMP22 gene can be associated with Charcot-Marie-Tooth neuropathy type IA (CMT1A) and hereditary neuropathy with liability to pressure palsies (HNPP). The methods of detecting CNVs described herein can be used to diagnose CNV disorders described herein and in publications incorporated by reference. The disease can be a disease described in Lupski J. (2007) Nature Genetics 39: S43-S47.

The methods provided herein can be used to detect fetal anueploides from a maternal sample, e.g., maternal blood sample, chorionic villus sample, or amniotic fluid. Aneuploides, e.g., fetal aneuploidies, can include, e.g., trisomy 13, trisomy 18, trisomy 21 (Down Syndrome), Klinefelter Syndrome (XXY), monosomy of one or more chromosomes (X chromosome monosomy, Turner's syndrome), trisomy X, trisomy of one or more chromosomes, tetrasomy or pentasomy of one or more chromosomes (e.g., XXXX, XXYY, XXXY, XYYY, XXXXX, XXXYY, XXXYY, XYYYY and XXYYY), triploidy (three of every chromosome, e.g. 69 chromosomes in humans), tetraploidy (four of every chromosome, e.g. 92 chromosomes in humans), and multiploidy. In some cases, an aneuploidy can be a segmental aneuploidy. Segmental aneuploidies can include, e.g., 1p36 duplication, dup(17)(p11.2p11.2) syndrome, Down syndrome, Pelizaeus-Merzbacher disease, dup(22)(q11.2q11.2) syndrome, and cat-eye syndrome. In some cases, an abnormal genotype, e.g., fetal genotype, is due to one or more deletions of sex or autosomal chromosomes, which can result in a condition such as Cri-du-chat syndrome, Wolf-Hirschhorn, Williams-Beuren syndrome, Charcot-Marie-Tooth disease, Hereditary neuropathy with liability to pressure palsies, Smith-Magenis syndrome, Neurofibromatosis, Alagille syndrome, Velocardiofacial syndrome, DiGeorge syndrome, Steroid sulfatase deficiency, Kallmann syndrome, Microphthalmia with linear skin defects, Adrenal hypoplasia, Glycerol kinase deficiency, Pelizaeus-Merzbacher disease, Testis-determining factor on Y, Azospermia (factor a), Azospermia (factor b), Azospermia (factor c), or 1p36 deletion. In some cases, a decrease in chromosomal number results in an XO syndrome. Methods for detecting fetal aneuploidy are described, e.g., in U.S. Pat. No. 8,293,470.

Excessive genomic DNA copy number variation can be found in Li-Fraumeni cancer predisposition syndrome (Shlien et al. (2008) PNAS 105:11264-9). CNV is associated with malformation syndromes, including CHARGE (coloboma, heart anomaly, choanal atresia, retardation, gential, and ear anomalies), Peters-Plus, Pitt-Hopkins, and thrombocytopenia-absent radius syndrome (see e.g., Ropers H H (2007) Am J of Hum Genetics 81: 199-207). The relationship between copy number variations and cancer is described, e.g., in Shlien A. and Malkin D. (2009) Genome Med. 1(6): 62. Copy number variations are associated with, e.g., autism, schizophrenia, and idiopathic learning disability. See e.g., Sebat J., et al. (2007) Science 316: 445-9; Pinto J. et al. (2010) Nature 466: 368-72; Cook E. H. and Scherer S. W. (2008) Nature 455: 919-923.

Copy number variations can be associated with resistance of cancer patients to certain therapeutics. For example, amplification of thymidylate synthase can result in resistance to 5-fluorouracil treatment in metastatic colorectal cancer patients. See Wang et al. (2002) PNAS USA vol. 99, pp. 16156-61. Methods of determining CNVs are described, e.g., in PCT Application Publication No. WO2012/109500.

In some cases, the methods, compositions, or kits provided herein are used to determine gene expression level (e.g., messenger RNA level). The expression level can be, for example, higher than normal, normal, or below normal. In some cases, the methods, compositions, and kits provided herein are used to determine if a sequence is mutated or wild-type, or has one or more mutations (e.g., a de novo mutation, nonsense mutation, missense mutation, silent mutation, frameshift mutation, insertion, substitution, point mutation, single nucleotide polymorphism (SNP), single nucleotide variant, de novo single nucleotide variant, deletion, rearrangement, amplification, chromosomal translocation, interstitial deletion, chromosomal inversion, loss of heterozygosity, loss of function, gain of function, dominant negative, or lethal).

Methods for analyzing nucleic acids, e.g., for detecting mutations, gene expression, or copy number variation, are described, e.g., in U.S. Patent Application Publication Nos. 20120252015, 2012021549, 20120214163, 20120225428, 20120245235, 20120252753, 20100196898, 20120270739, 20110171646, and U.S. Pat. Nos. 8,304,194

Computer Systems

The methods described herein can be implemented by one or more computer systems. Computer systems can include various combinations of a computer processor (also “processor” herein) or other processing device, an internal communication bus, various types of memory or storage media (RAM, ROM, EEPROM, cache memory, disk drives, etc.) for code and data storage, and one or more network interface cards or ports for communication purposes. The methods described herein can include or be implemented in software comprising machine-executable code, which can run on such computer systems or other systems. For example, the software can be executable by a computer system, for example, that functions as the storage server or proxy server, and/or that functions as a user's terminal device. During operation the code can be stored within the computer system, such as in a memory location (e.g., memory, hard disk, cache) of the computer system. At other times, the code can be stored at other locations and/or transmitted for loading into the appropriate computer system. Execution of the code by a processor of the computer system can enable the computer system to implement the methods described herein.

In some cases, software can be stored on a computer system in the form of a non-transitory computer readable medium. The non-transitory computer readable medium can have stored therein sequences of instructions which, when executed by a computer system, cause the computer to perform methods described herein. Computer readable media can include, e.g., a hard disk, diskette, random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), CD±R, CD±RW, DVD, DVD±RW, DVD±R, DVD-RAM, HD DVD, HD DVDR, HD DVD±RW, HD DVD±RAM, Blu-ray Disc, optical or magnetic storage medium, paper tape, punch cards, optical mark sheets or any other device that is capable of providing data or executable instructions that can be accessed by a processing system. Computer readable medium are described, e.g., in U.S. Pat. No. 7,783,072.

A computer can be in communication with a device, e.g., a thermocycler or a device for performing droplet digital PCR. A computer can be connected to the Internet through a wired or wireless connection.

In some cases, a health care provider or subject sends a sample to a service provider that analyzes the sample using the methods, compositions, or kits described herein. In some cases, a computer is used to transmit results of a reaction to a subject. In some cases, the subject is a patient. In some cases, a computer is used to transmit results of a reaction to a healthcare provider, e.g., a physician, or to an insurance company. In some cases, a computer is used to generate a report comprising results of one or more reactions and/or additional assays.

While some embodiments have been shown and described herein, it will be obvious to those skilled in the art that such embodiments are provided by way of example only. Numerous variations, changes, and substitutions will now occur to those skilled in the art. It should be understood that various alternatives to the embodiments described herein can be employed. It is intended that the following claims define the scope and that methods and compositions within the scope of these claims and their equivalents be covered thereby.

1. A method for amplifying a target polynucleotide, comprising:
(a) contacting a target polynucleotide with a reaction mixture comprising:
(i) a first forward primer that is complementary to a first sequence of said target polynucleotide and comprises: a first modified nucleoside residue at a position corresponding to a target locus residing within said first sequence, a first fluorophore that is attached to said first forward primer and is upstream of said target locus, and a first quencher that is attached to said first forward primer and is downstream of said target locus;
(ii) a first reverse primer; and
(iii) a digestive enzyme that is capable of cleaving said first forward primer when said modified nucleoside residue matches a first allele at said target locus, and is not capable of cleaving said first forward primer when said modified nucleoside residue does not match said first allele at said target locus; and
(b) amplifying said target nucleotide in said reaction mixture with a DNA polymerase, thereby obtaining a plurality of amplicons.

2. The method of claim 1, wherein said first modified nucleoside comprises a ribonucleoside.

3. The method of claim 1, wherein said first modified nucleoside comprises a 2′-fluoro-modified RNA nucleoside.

4. The method of claim 1, wherein said first modified nucleoside comprises a 2′-fluoro-modified DNA nucleoside.

5. The method of claim 1, wherein said first forward primer further comprises a blocking residue located downstream of said target locus.

6. The method of claim 5, wherein said blocking residue comprises an inverted base.

7. The method of claim 5, wherein said blocking residue locates about 1 to 10 bases from said target locus.

8. The method of claim 5, wherein said blocking residue locates between said target locus and the residue to which said quencher is attached.

9. The method of claim 7, wherein said blocking residue locates about 1 to 10 bases from said quencher.

10. The method of claim 1, wherein said digestive enzyme comprises RNase H2.

11. The method of claim 1, wherein said digestive enzyme is thermostable.

12. The method of claim 1, wherein said DNA polymerase substantially lacks 5′-nuclease activity.

13. The method of claim 1, further comprising detecting said amplicons.

14. The method of claim 13, wherein said detecting is carried out in real-time.

15. The method of claim 13, wherein said detecting comprises a melting curve analysis.

16. The method of claim 1, wherein said amplifying step is carried out by performing digital PCR.

17. The method of claim 16, wherein said digital PCR is microfluidic-based digital PCR.

18. The method of claim 16, wherein said digital PCR is droplet digital PCR.

19. The method of claim 16, wherein said digital PCR is performed in droplets having a volume that is between about 1 pL and about 100 nL.

20. The method of claim 1, wherein said first modified residue matches said first allele.

21. The method of claim 1, wherein said first modified residue does not match said first allele.

22. The method of claim 1, wherein said first reverse primer comprises
(i) a modified residue, and
(ii) a blocking group.

23. The method of claim 1, wherein said reaction mixture comprises a second forward primer comprising: a second modified nucleoside residue at a position corresponding to said target locus, a second fluorophore that is attached to said second forward primer and is upstream of said target locus, and a second quencher that is attached to said second forward primer and is downstream of said target locus, wherein said second modified nucleoside residue matches a second allele of said target locus.

24. The method of claim 1, wherein said reaction mixture comprises two to four forward primers, each comprising: a modified nucleoside residue at a position corresponding to said target locus, a fluorophore that is attached to each of said forward primers and is upstream of said target locus, and a quencher that is attached to each of said forward primers and is downstream of said target locus, wherein said modified nucleoside residue of each of said forward primers matches a different allele of said target locus.

25. The method of claim 13, wherein said detecting has a sensitivity of 1/100 to 1/1,000, as defined by mutant/(mutant+wild-type).

26. A primer that is complementary to a first sequence of a target polynucleotide, comprising:
(a) a first modified nucleoside residue at a position corresponding to a target locus residing within said first sequence;
(b) a fluorophore attached to said primer and is upstream of said target locus; and
(c) a quencher attached to said first primer and is downstream of said target locus.

27. The primer of claim 26, wherein said first modified nucleoside comprises a ribonucleoside.

28. The primer of claim 26, wherein said first modified nucleoside comprises a 2′-fluoro-modified RNA nucleoside.

29. The primer of claim 26, wherein said first modified nucleoside comprises a 2′-fluoro-modified DNA nucleoside.

30. The primer of claim 26, wherein said first forward primer further comprises a blocking residue located downstream of said target locus.

31. The primer of claim 30, wherein said blocking residue comprises an inverted base.

32. The primer of claim 30, wherein said blocking residue locates about 1 to 10 bases from said target locus.

33. The primer of claim 30, wherein said blocking residue locates between said target locus and the residue to which said quencher is attached.

34. The primer of claim 33, wherein said blocking residue locates about 1 to 10 bases from said quencher.

35. (canceled)
